(19) United States
(12) Patent Application Publication
(10) Pub. No.: US 2005/0118226 A1
(43) Pub. Date: Jun. 2, 2005

(54) Methods for treating ACAT-related diseases

(76) Inventors: Dora M. Kovacs, Charlestown, MA (US); Luigi Puglielli, Madison, WI (US); Rudolph E. Tanzi, Hull, MA (US); Matthew P. Frosch, Charlestown, MA (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/983,969

(22) Filed: Nov. 8, 2004

Related U.S. Application Data
(60) Provisional application No. 60/518,492, filed on Nov. 7, 2003.

(51) Int. Cl. A61K 31/4965; A61K 9/22

(52) U.S. Cl. 424/423; 604/890.1

(57) ABSTRACT

The invention relates to methods for administering inhibitors of acyl-coenzyme A:cholesterol acyltransferase (ACAT) activity, and for treating Alzheimer's disease, atherosclerosis, and other ACAT-related diseases. The invention also relates to sustained release delivery systems.

### RELATED APPLICATIONS

This application claims the benefit under 35 U.S.C. § 119(e) of U.S. provisional application Ser. No. 60/518,492, filed Nov. 7, 2003, the disclosure of which is incorporated by reference herein.

### FIELD OF THE INVENTION

The invention relates in part to methods for administering inhibitors of acyl-coenzyme A:cholesterol acyltransferase (ACAT) activity, and for treating Alzheimer's disease, atherosclerosis, and other diseases.

### BACKGROUND OF THE INVENTION

Alzheimer's disease (AD) is a neurodegenerative disorder characterized by the abnormal deposition of insoluble protein aggregates in cortical brain regions. Senile plaques constitute the majority of extracellular deposits, and are mainly composed of the amyloid β-peptide (Aβ) (Glenner et al., 1984). Aβ is a 39-43 amino acid hydrophobic polypeptide, proteolytically derived from a much larger precursor, the amyloid precursor protein (APP) (Kang et al., 1987; Tanzi et al., 1987). For Aβ biogenesis, APP is first cleaved at the N-terminus of Aβ (β-cleavage) by β-site APP cleaving enzyme 1 (BACE1), producing a 99 amino acid C-terminal fragment (C99) that migrates with the apparent molecular mass of 12-kDa. C99 is subsequently cleaved in the transmembrane domain (γ-cleavage by the presenilin-dependent γ-secretase) releasing Aβ (Aguzzi and Haass, 2003; Citron, 2004; De Strooper, 2003; Sinha et al., 1999; Vassar, 2004; Xia and Wolfe, 2003). Alternatively, α-secretase cleaves in the middle of the Aβ region of APP preventing Aβ generation (Ling et al., 2003). The two major sites of γ-cleavage are located at positions 40 and 42 of Aβ, generating Aβ40 and Aβ42, respectively. Normally, the 40 amino acid variety represents 90% of secreted Aβ (Selkoe, 1999).

In the last few years, cholesterol metabolism has been established as a risk factor for AD by genetic (Sing and Davignon, 1985; Ehnholm et al., 1986; Boerwinkle et al., 1987; Corder et al., 1993; Schmechel et al., 1993), epidemiological (Jarvik et al., 1995; Kuo et al., 1998; Notkola et al., 1998; Koudinov et al., 1998; Jick et al., 2000; Wolozin et al., 2000), and biochemical (Refolo et al., 1991; Lee et al., 1998; Parkin et al., 1999) studies. In addition, both animal (Refolo et al., 2000; Refolo et al., 2001; Fassbender et al., 2001) and cellular (Frears et al., 1999; Fassbender et al., 2001; Puglielli et al., 2001) models of AD have shown that cholesterol homeostasis and distribution regulate APP processing and Aβ generation (for a review, see Puglielli et al., 2003).

Thus, genetic and epidemiological data support a role for altered cholesterol metabolism in the pathogenesis of AD (Burns and Duff, 2002; Hartmann, 2001; Puglielli et al., 2003). Statins, which inhibit cholesterol generation and internalization and thereby lower total cholesterol in cells, reduce Aβ production in cell cultures and in most animal models of AD (Petanceska et al., 2002). In retrospective analysis of clinical trials, statins lowered the risk of the onset of AD by up to 73% (Jick et al., 2000; Wolozin et al., 2000). Cognition in AD patients improved slightly in the first small prospective trial with simvastatin (Simons et al., 2002) and large clinical trials are ongoing (Wolozin, 2004). Since patients affected by AD often exhibit normal cholesterol levels, the question is whether statins will be able to provide significant cognitive benefits for these subjects. Accordingly, additional types of therapeutics for the treatment of AD would be beneficial.

An alternative approach to statins, which lower total cholesterol levels in cells, is to alter intracellular cholesterol homeostasis. Acyl-coenzyme A: cholesterol acyltransferase (ACAT) is an endoplasmic reticulum resident membrane protein that generates cholesteryl-esters from free membrane cholesterol and fatty acids. The ACAT1 isoform of the enzyme regulates intracellular cholesterol homeostasis by converting excess membrane cholesterol into cytoplasmic cholesteryl-ester droplets (Chang et al., 2001). These lipid droplets are in turn can be hydrolyzed when more free cholesterol and fatty acids are needed. ACAT inhibition has long been studied as a potential antiatherosclerotic strategy, resulting in both reduced intestinal cholesterol absorption and foam cell formation (Shah, 2003).

It previously has been shown that inhibition of ACAT activity results in reduced Aβ generation in cell culture models of AD and in primary neurons (Puglielli et al., 2001). In that work, it was shown that two ACAT inhibitors, CP-113,818 and Dup128, inhibited Aβ production by up to 50%. However, ACAT inhibitors are known to by cleared from the circulation quickly in animals, which renders them unsuitable for therapeutic use for altering the ratio of free cholesterol to cholesterol esters. Indeed, a previous study had reported 1000-fold fluctuations in blood CP-113,818 levels when the inhibitor was orally administered to monkeys (Marzetta et al., 1994).

Therefore, suitable therapeutic approaches involving inhibition of ACAT activity to alter intracellular cholesterol homeostasis still are needed.

### SUMMARY OF THE INVENTION

We have determined that administration of ACAT inhibitor using a sustained release delivery system (an implant) surprisingly provides effective inhibition of ACAT in a manner that enables the effective treatment of ACAT-related diseases. We have used the ACAT inhibitor CP-113,818 in a transgenic mouse model to assess whether ACAT inhibitors can be delivered in a manner to effectively inhibit ACAT, thereby making ACAT a potential target for anti-amyloid therapy in patients affected by AD and other ACAT-related diseases. hAPP mice harbor a human APP751 transgene with the Swedish double and London mutation under the neuronal Thy1 promoter (Rockenstein et al., 2001). The mice undergo AD-like neurodegeneration, with clear memory deficits at 6 months of age. Plaques are detectable in the neocortex and hippocampus at the ages of 4 and 6 months, respectively. In the current studies, we administered the ACAT inhibitor CP-113,818 starting at 5 months of age for 2 months. Our results provide evidence that ACAT can be effectively inhibited when administered using a sustained release formulation, and for the potential usefulness of ACAT inhibitors in the prevention and treatment of AD as well as other ACAT-related diseases.

According to one aspect of the invention, methods for treating an ACAT-related disease in a subject are provided. The methods include administering an ACAT inhibitor using a sustained release delivery system to a subject that has or is suspected of having an ACAT-related disease. The sustained-release implant releases an amount of the ACAT inhibitor for a time that is effective to reduce ACAT activity and thereby treat the ACAT-related disease. In some embodiments, the ACAT inhibitor is administered for at least one month, preferably for at least two months, and more preferably for at least three months.

ACAT-related diseases include diseases in which amyloid β is elevated, such as Alzheimer's disease, and atherosclerosis.

In some embodiments, the sustained release delivery system is a sustained release implant that is implanted in the subject. In other embodiments, the sustained release delivery system is a pump-based delivery system, preferably one that is implanted in the subject.

In still other embodiments, the methods also include administering a second therapeutic agent for treating the ACAT-related disease. Such second therapeutic agents include statin molecules and secretase inhibitor molecules. The second therapeutic agent can be co administered using the sustained release delivery system.

In another aspect of the invention, methods for reducing the amount of amyloid-β in a subject are provided. The methods include administering an ACAT inhibitor using a sustained release delivery system to the subject. The sustained release delivery system releases an amount of the ACAT inhibitor for a time that is effective to reduce the amount of amyloid-β in the subject. In some embodiments, the subject has or is suspected of having elevated levels of amyloid-β, amyloid-β-containing plaque load and/or insoluble amyloid-β. In certain of these embodiments, the methods provide reduction of amyloid-β in neural tissues of the subject. In some embodiments, the ACAT inhibitor is administered for at least one month, preferably for at least two months, and more preferably for at least three months.

In further embodiments, the subject has or is suspected of having Alzheimer's disease.

The reduction in amyloid-β in the subject in some embodiments reduces amyloid-β-containing plaque load. The reduction in amyloid-β levels can be determined by a diagnostic imaging method.

In some embodiments, the sustained release delivery system is a sustained release implant that is implanted in the subject. In other embodiments, the sustained release delivery system is a pump-based delivery system, preferably one that is implanted in the subject.

In still other embodiments, the methods also include administering a second therapeutic agent for treating the ACAT-related disease. Such second therapeutic agents include statin molecules and secretase inhibitor molecules. The second therapeutic agent can be co administered using the sustained release delivery system.

In a third aspect of the invention, a sustained release delivery system is provided that is configured to contain and deliver to a subject an effective amount of an ACAT inhibitor to inhibit ACAT activity in the subject. In certain embodiments, the effective amount of the ACAT inhibitor reduces ACAT activity in neural tissues and/or reduces amyloid-β levels.

The sustained release delivery system is in some embodiments an implant or a pump-based system, preferably an implantable pump-based system.

The delivery system is in some embodiments configured to contain and deliver to a subject an effective amount of a second therapeutic agent for treating an ACAT-related disease. The second therapeutic agent is preferably a statin molecule and/or a secretase inhibitor molecule.

These and other aspects of the invention will be described in further detail in connection with the detailed description of the invention.

### BRIEF DESCRIPTION OF THE DRAWINGS

FIG. 1 shows that CP-113,818 inhibits cholesteryl-ester generation in mouse brains without inducing adrenal toxicity.

FIG. 1A: Concentration-dependent inhibition of ACAT activity in non-transgenic mice treated for 21 days with CP-113,818. The ACAT inhibitor was most effective at 7.1 mg/kg/day, reducing total cholesterol in the serum by 29% and cholesteryl-ester levels in the brain and liver by 86% and 93%, respectively, when compared to placebo-treated animals. Free or membrane cholesterol was decreased by up to 37% in the liver. Data represent mean+/−SD, n=3 (Student's 2-tailed t test, *p<0.05, **p<0.01, ***p<0.001).

FIG. 1B: hAPP mice treated with 7.2 mg/kg per day of CP-113,818 for two months did not show signs of vacuolization in the cytoplasm of adrenal cortical cells (lower panels), which would represent a first indication of toxicity derived from most ACAT inhibitors.

FIG. 1C: Decreased levels of serum (35%, p<0.0003) and liver cholesterol (34%, p<0.00009), and liver cholesteryl-esters (87%, p<0.00001) in hAPP mice treated with 7.2 mg/kg/day of CP-113,818 for two months (57 days; n=6/treatment). These determinations were made on the same animals that were also used for brain amyloid and behavior studies. Data represent mean+/−SD, n=6 (Student's 2-tailed t test, ***p<0.001).

FIG. 2 shows the brain and neuronal cholesterol distribution in non-transgenic mice. Free (or membrane) cholesterol levels are relatively high in total brain extracts due to cholesterol immobilized in myelin membranes (upper panel). In contrast, when the neuronal population is enriched during the course of preparing primary neuronal cultures, myelin membranes are largely discarded and the ratio between free cholesterol and cholesteryl-esters decreases (lower panel). Note that the enriched neuronal preparation was assayed for cholesterol levels prior to being placed into media containing exogenous lipid sources.

FIG. 3 demonstrates that CP-113,818 treatment for 57 days markedly reduces amyloid load in the brains of hAPP transgenic mice.

FIG. 3A: Representative amyloid staining (using thioflavin S) and Aβ staining (using the antibody 6E10) of female hAPP mouse cortices at 6.5 months of age following placebo and CP-113,818 treatments. Aβ staining of cortices at day 0 (at 4 months of age) without and, at day 57, with CP-113,818 treatment was remarkably similar, suggesting that the ACAT inhibitor prevented formation of amyloid pathology in these mice.

FIG. 3B: Quantitative analysis of brain plaque load, as assessed by number of plaques per μm² stained with the Aβ-specific antibody 6E10. In the hAPP animal model, the cortex is the first to develop amyloid pathology, followed by the hippocampus. Therefore, at 6.5 months of age, males exhibit distinct AD pathology in the cortex but not in the hippocampus. CP-113,818 reduced cortical plaque number by ˜88%, equally in females (89%) and males (88%). The most marked effect was observed in the hippocampus of female mice, where amyloid load was decreased by 99%, p<0.000001.

FIG. 4 shows that CP-113,818 treatment reduces Aβ staining and amyloid burden in the brains of hAPP mice.

FIG. 4A: Representative Aβ stainings in the cortex and hippocampus of female and male hAPP mice, as assessed by staining with the Aβ-specific antibody 6E10. These images are quantitatively expressed in FIG. 2 and the “B” section of this figure. While Aβ deposition in placebo-treated animals is more pronounced in females as compared to males, CP-113,818 decreased Aβ staining to the same levels.

FIG. 4B: Plaque burden calculated as the percent of area covered by plaques in a particular brain region. CP-113,818 decreased plaque burden by up to 97%, in a gender-independent manner (see cortex, values calculated by gender).

FIG. 5 shows that CP-113,818 treatment reduces levels of insoluble and soluble Aβ₁₋₄₀ and Aβ₁₋₄₂ in the brains of hAPP transgenic mice.

FIG. 5A: Sandwich ELISAs of insoluble Aβ₁₋₄₀ and Aβ₁₋₄₂ in brain formic acid extracts revealed a significant decrease of both peptides in CP-113,818-treated animals (92% and 83%, respectively). The significance was higher in females than in males, due to lower starting Aβ levels in brain extracts of placebo-treated males.

FIG. 5B: Sandwich ELISAs of soluble Aβ₁₋₄₀ and Aβ₁₋₄₂ in TBS fractions lacking detergents revealed a decrease of both peptides in CP-113,818-treated animals (12% and 34%, respectively), which was significant for Aβ₁₋₄₂. Aβ₁₋₄₀ levels were close to the baseline of the assay. A decrease in both male and female animals was observed.

FIG. 6 shows that CP-113,818 treatment restores normal spatial learning and memory in female hAPP mice in a Morris water maze test. Behavioral performance assessing spatial navigation was evaluated for each animal three times/day for four days, between days 54 and 57 of treatment with CP-113,818. In each MWM trial, swimming path (length in meters required to reach the platform) and escape latency (seconds to reach the platform) were measured. The top two panels show data collected from all animals in our study, regardless of gender. In addition to hAPP transgenic mice, we also used equal numbers of non-transgenic littermates untreated and treated with CP-113,818 in this study. The results show only a slight impairment in MWM trials in transgenic mice when compared to non-transgenics, thus leaving little room for improvement due to reduced amyloid accumulation by CP-113,818. The lower panels show exclusively results obtained with female mice. Correlating with two-fold increased amyloid load in the female relative to the male cohort, only female transgenic animals exhibited a significant decline in performance when compared to non-transgenic littermates. Treatment with CP-113,818 improved this performance in the female cohort (p<0.014 on day 3), so that treated hAPP transgenic animals performed similarly to non-transgenics in both the swimming path and escape latency tests. Data represent mean+/−SEM, n=3 trials/animal/day, statistical differences were calculated with the Mann-Whitney U-test.

FIG. 7 shows that CP-113,818 treatment does not affect normal spatial learning and memory in male hAPP mice in a Morris water maze test. Correlating with limited amyloid load at 6.5 months of age in male hAPP mice, no significant decline in performance was observed in this cohort when compared to non-transgenic littermates. Consistently, treatment with CP-113,818 did not affect the performance in the male cohort. Note that swimming path of male non-transgenic animals was slightly improved by CP-113,818 treatment. However, this was not reflected in escape latency and was not observed in hAPP mice, suggesting that potential cholesterol-mediated effects of CP-113,818 may not explain this slight change. Data represent mean+/−SEM, n=3 trials/animal/day, statistical differences were calculated with the Mann-Whitney U-test.

FIG. 8 indicates that synaptophysin immunoreactivity in the CA1 layer of the hippocampus is unchanged following treatment with CP-113,818. Representative immunostaining (FIG. 8A) and quantification of the staining in female and male animals (FIG. 8B) did not reveal differences in synaptic density between placebo- and CP-113,818-treated animals. However, the animals used in this study were too young to show changes in synaptic density induced by the APP transgene when transgenic and non-transgenic animals were compared (data not shown). Sto (stratum oriens), stp (stratum pyramidale), str (stratum radiale)

FIG. 9 shows that levels of formic acid-extracted sAPP are decreased by CP-113,818 treatment of hAPP mice.

FIG. 9A: Representative Western blot of full-length APP and its C-terminal fragments C99 and C83 (antibody: C7), and of secreted APP (antibody: 22C11), obtained from formic acid extracts (left). Densitometric analysis of APP fragments in each lane, adjusted to the total amount of full-length APP in the same lane, showed CP-113,818 to decrease sAPP levels by 60%.

FIG. 9B: Densitometric analysis of APP fragments resolved on Western blots, as described above, shown separately for each animal in the study. Statistical significance was determined by Student's t test.

FIG. 10 shows that CP-113,818 treatment of non-transgenic littermates for 57 days reduces processing of endogenous APP, but not Notch or N-cadherin, without directly inhibiting β- and γ-secretase activities or Aβ aggregation.

FIG. 10A: Representative Western blots of APP and its cleavage products from non-transgenic mouse brains after 60 days of treatment with CP-113,818. Full-length APP (APP-FL) and its C-terminal fragments (APP-CTF) were detected in brain lysates, while total secreted APP (sAPP) and secreted APPα (sAPP-α) in the initial soluble TBS fraction lacking detergents. Treatment with CP-113,818 reduces the amounts of APP-CTFs as well as total sAPP (which includes sAPP-β), while sAPP-α only shows a trend toward decrease.

FIG. 10B: Densitometric analysis of APP fragments resolved on Western blots. APP C-terminal fragments (CTFs) were normalized to full-length APP whereas sAPPₜₒₜₐₗ is shown alone and also as a ratio between sAPPₜₒₜₐₗ and sAPPα, to better illustrate a reduction in sAPP forms other than sAPPα, such as sAPPβ. Statistical significance (n=8, p<0.05) was determined by Student's t test.

FIG. 10C: Representative Western blots of γ-secretase complex components and substrates after treatment of non-transgenic littermates with CP-113,818. CP-113,818 did not alter maturation of γ-secretase complex in vivo, as shown by unchanged glycosylation of nicastrin, or levels of presenilin N- and C-terminal fragments (PS 1-NTF and CTF), nicastrin, or pen-2. Furthermore, levels of N-cadherin C-terminal fragment 1 (CTF) and Notch-1 intracellular domain (ICD) remained unchanged suggesting that treatment with CP-113,818 does not perturb normal functions of the γ-secretase complex. *: aspecific band.

FIG. 10D: Representative Western blots of BACE1 and ApoE after treatment of non-transgenic littermates with CP-113,818. Treatment with CP-113,818 did not alter expression levels of BACE1 in brain lysates or ApoE in the soluble TBS fraction.

FIG. 10E: In vitro fluorometric BACE1 activity assay performed with purified recombinant human BACE1 in the presence of increasing amounts of CP-113,818. While GL-189, a specific β-secretase inhibitor, significantly reduces BACE1 activity, CP-113,818 has no direct effect on BACE1 activity in vitro.

FIG. 10F: In vitro γ-secretase activity assay performed on CHO cell (overexpressing APP) membrane fractions in the presence of increasing amounts of CP-113,818. While L-685,458, a specific γ-secretase inhibitor, inhibits APP intracellular domain (AICD) generation, CP-113,818 has no direct effect on γ-secretase activity in vitro. C99 and C83: APP C-terminal fragments.

FIG. 10G: In vitro aggregation assay testing the effect of CP-113,818 on metal- or Aβ₄₂-mediated aggregation of Aβ₄₀. Aβ₄₀ was incubated alone or with “aged” Aβ₄₂ or Zn⁺⁺ with or without 10 μM CP-113,818 in the wells of a 384-well microassay plate. Sample turbidity was monitored by following well absorbance at 400 nm over four days. Test signal was blanked on wells containing no Aβ₄₀. CP-113,818 does not directly modulate metal- or Aβ₄₂-mediated aggregation of Aβ₄₀ in vitro. Aggregation of Aβ₄₀ alone was not significant (p<0.671 between time 0 and 4 days). Data points are shown as average of eight replicates±standard error. Significance values were calculated using students t-test (2-tailed). (Gray solid square: Aβ₄₀; black unfilled triangle: Aβ₄₀+CP-113,818; gray solid triangle: Aβ₄₀+Aβ₄₂; black unfilled circle: Aβ₄₀+Aβ₄₂+CP-113,818; gray solid circle: Aβ₄₀+Zn; black unfilled square: Aβ₄₀+Zn⁺ CP-113,818).

### DETAILED DESCRIPTION OF THE INVENTION

It has been discovered that acyl-coenzyme A:cholesterol acyltransferase (ACAT) inhibitor compounds, in order to be effective in reducing the amount of amyloid β (Aβ) produced, must be administered to a subject for an extended and sustained period of time. Similarly, sustained release administration of ACAT inhibitors can be used in the treatment of other ACAT-related diseases, such as atherosclerosis.

The methods of the invention include administering ACAT inhibitor compounds using a sustained release delivery system, e.g., an implant, for treating Alzheimer's disease and other diseases that are related to cholesterol ester accumulation by the action of ACAT, which are termed herein as “ACAT-related” diseases or disorders. The implant provides ACAT inhibitor compounds for a sustained period of time to inhibit constantly and effectively ACAT activity and thereby reduce Aβ production. One set of such ACAT-related diseases involve the accumulation of Aβ, which are referred to herein as “Aβ-associated” disorders. As used herein, the term “Aβ-associated disorder” and the like includes, but is not limited to: Alzheimer's disease, Down's syndrome, cerebrovascular amyloidosis, hereditary amyloidosis with cerebral hemorrhage of the Dutch type, vascular dementia, and inclusion body myositis. Other ACAT-related diseases or disorders include diseases related to excess cholesterol accumulation, such as atherosclerosis, and diseases caused by deleterious alteration of intracellular cholesterol homeostasis, such as the excessive formation of cytoplasmic cholesteryl-ester droplets.

As used herein, an “ACAT inhibitor” or a “compound that inhibits ACAT” is an agent, such as a specific molecule or combination of molecules, that inhibit ACAT activity by inhibition of enzymatic activity or expression of ACAT. Compounds that inhibit ACAT include: small molecule compounds which typically are organic chemical compounds, RNAi and/or siRNA oligonucleotides that hybridize to ACAT-encoding nucleic acid molecules, and ACAT antibodies. Such compounds preferably will reduce ACAT activity by at least about 1.0%, 2.0%, 3.0%, 4.0%, 5.0%, 7.0%, 10%, 15%, 20%, 25%, 30%, 40%, 50%, or more. These and other compounds may be administered alone or in combination as part of a pharmaceutical composition. The combinations can include one or more ACAT inhibitors, optionally with other therapeutics such as statins.

ACAT inhibitors encompass numerous chemical classes, although typically they are organic compounds. In some embodiments, the ACAT inhibitors are small organic compounds, i.e., those having a molecular weight of more than 50 yet less than about 2500, preferably less than about 1000 and, more preferably, less than about 500. The ACAT inhibitors include functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The ACAT inhibitors can include cyclic carbon or heterocyclic structures and/or aromatic or polyaromatic structures substituted with one or more of the above-identified functional groups. ACAT inhibitors also can be biomolecules such as peptides, saccharides, fatty acids, sterols, isoprenoids, purines, pyrimidines, derivatives or structural analogs of the above, or combinations thereof and the like. Where the ACAT inhibitors is a nucleic acid molecule, the agent typically is a DNA or RNA molecule, although modified nucleic acid molecules as defined herein are also contemplated.

ACAT inhibitors can be obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents can be tested and further may be subjected to directed or random chemical modifications such as acylation, alkylation, esterification, amidification, etc. to produce structural analogs of the agents.

Presently known ACAT inhibitors include CP 113,818 (Pfizer); NTE-122, [(trans-1,4-bis[[1-cyclohexyl-3-(4-dimethylamino phenyl)ureido]methyl]cyclohexane)] (Nissin Food Products Co., Ltd); F-1394, [(1S,2S)-2-[3-(2,2-dimethylpropyl)-3-nonylureido] cyclohexane-1-yl 3-[(4R)-N-(2,2,5,5-tetramethyl-1,3-dioxane-4-carbonyl)amino]propionate)] (Fujirebio Inc.); avasimibe (C₁-1011) (Pfizer); PD 140296 (Parke-Davis); PD 128042 [2,2-dimethyl-N-(2,4,6-trimethoxyphenyl)-dodecanamide] (Parke-Davis); PD 132301-2 (Parke-Davis); octimibate, (8-[1,4,5-triphenyl-1H-imidazole-2-yl)-oxy]octanoic acid); CI 976 (2,2-dimethyl-N-(2,4,6-trimethoxyphenyl)dodecanamide); DuP 128 (N′-(2,4-difluorophenyl)-N-[5-(4,5-diphenyl-1H-imidazol-2-ylthio)pentyl]-N-heptylurea); 58-035 [3-(decyldimethyl-silyl)N-[2-(4-methyl-phenyl)1-phenylethyl propanamide] (Sandoz); HL-004 [N-(2,6-diisopropylphenyl)tetradecylthioacetamide]; SMP-500 (Sumitomo Pharmaceuticals Co.); CL-277,082 [2,4-difluoro-phenyl-N[[4-(2,2-dimethylpropyl)phenyl]methyl]-N-(hepthyl)urea]; SKF-99085 (Glaxo Smith-Kline); CS-505 (Sankyo Pharma); Eflucimibe, S)-2-(Dodecylthio)-4′-hydroxy-2′,3′,5′-trimethyl-2-phenylacetanilide (bioMérieux-Pierre Fabre/Eli Lilly); F 12511; E5324 (n-butyl-N′-[2-[3-(5-ethyl-4-phenyl-1H-imidazol-1-yl)propoxy]-6-methylphenyl]urea); R-755 (N-(2,6-diethylphenyl)-N′-[3-(2-methylphenyl)-6,7-dihydro-5H-cyclopentaμf[1]benzothiophen-2-yl]urea); FR145237 (Fujisawa Pharmaceutical Co., Ltd.); CL277,082; YM-17E, 1,3-bis[[1-cycloheptyl-3-(p-dimethylamino-phenyl)ureido]methyl]benzene dihydrochloride; FR129169 (N-(1,2-diphenylethyl)-2-octyloxyphenylacetamide) (Fujisawa Pharmaceutical Co., Ltd.); tamoxifen (J. Pharmacol Exp. Ther. 2004, 308(3):1165-1173); and analogs thereof.

Other ACAT inhibitors include 1) contacting a cell with nucleic acid molecules that are antisense of the nucleic acids that encode ACAT and that inhibit ACAT expression, 2) RNAi and/or siRNA molecules that inhibit ACAT expression, and 3) antibodies that block the functional activity of ACAT. Additional ACAT inhibitors include polypeptides that are variants of the ACAT molecules that are not functional or are not fully functional (and nucleic acids that encode such polypeptides). Such variants may compete with the functional endogenous ACAT molecules in a cell, tissue, or subject, and thereby reduce the ACAT activity.

The antisense oligonucleotides, RNAi, or siRNA nucleic acid molecules used for this purpose may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art-recognized methods, which may be carried out manually or by an automated synthesizer. They also may be produced recombinantly by vectors.

The antisense or siRNA nucleic acid molecules also may include “modified” oligonucleotides. That is, the oligonucleotides may be modified in a number of ways, which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars that are covalently attached to low molecular weight organic groups other than a hydroxyl group at the 3′ position and other than a phosphate group at the 5′ position. Thus, modified oligonucleotides may include a 2′-O-alkylated ribose group. In addition, modified oligonucleotides may include sugars such as arabinose instead of ribose.

The invention provides ACAT inhibitors in a sustained release delivery system. In addition to providing a constant level of therapeutic (i.e., ACAT inhibitor), the sustained release system provides avoids repeated administrations of the therapeutic agent of the invention, increasing convenience to the subject and the physician, as well as compliance with dosage regimen. The therapeutic compositions may be prepared by any of the methods well known in the art of pharmacy. All methods include the step of bringing the compounds into association with a carrier that constitutes one or more accessory ingredients. In general, the compositions are prepared by uniformly and intimately bringing the therapeutic agent into association with a carrier (e.g., a polymer composition constituting an implant), and then, if necessary, shaping the product.

Many types of sustained release delivery systems are available and known to those of ordinary skill in the art. They include polymer-based systems such as polylactic and polyglycolic acid, poly(lactide-glycolide), copolyoxalates, polyanhydrides, polyesteramides, polyorthoesters, polyhydroxybutyric acid, and polycaprolactone. Microcapsules of the foregoing polymers containing drugs are described in, for example, U.S. Pat. No. 5,075,109. Nonpolymer systems that are lipids including sterols such as cholesterol, cholesterol esters and fatty acids or neutral fats such as mono-, di- and tri-glycerides; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

In one particular embodiment, the preferred vehicle is a biocompatible microparticle or implant that is suitable for implantation into the mammalian recipient. Exemplary bioerodible implants that are useful in accordance with this method are described in PCT International application no. PCT/US95/03307 (Publication No. WO 95/24929, entitled “Polymeric Gene Delivery System”. PCT/US95/03307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the compound(s) of the invention is encapsulated or dispersed within the biocompatible, preferably biodegradable polymeric matrix disclosed in PCT/US95/03307.

The polymeric matrix can be in the form of an implant wherein the compound is dispersed throughout a solid polymeric matrix. The polymeric matrix also can be in the form of a microparticle such as a microsphere (wherein the compound is dispersed throughout a solid polymeric matrix) or a microcapsule (wherein the compound is stored in the core of a polymeric shell). Other forms of the polymeric matrix for containing the compounds of the invention include films, coatings, gels, and stents. The size and composition of the polymeric matrix device is selected to result in favorable release kinetics in the tissue into which the matrix device is implanted. The size of the polymeric matrix device further is selected according to the method of delivery that is to be used. The polymeric matrix composition can be selected to have both favorable degradation rates and also to be formed of a material that is bioadhesive, to further increase the effectiveness of transfer when the device is administered to a vascular surface. The matrix composition also can be selected not to degrade, but rather, to release by diffusion over an extended period of time.

Both non-biodegradable and biodegradable polymeric matrices can be used to deliver agents of the invention of the invention to the subject. Biodegradable matrices are preferred. Such polymers may be natural or synthetic polymers. Synthetic polymers generally are preferred. The polymer is selected based on the period of time over which release is desired, generally in the order of a few hours to a year or longer. Typically, release over a period ranging from between a few weeks and three to twelve months is most desirable. The polymer optionally is in the form of a hydrogel that can absorb up to about 90% of its weight in water and further, optionally is cross-linked with multi-valent ions or other polymers.

In general, the agents of the invention are delivered using the bioerodible implant by way of diffusion, or more preferably, by degradation of the polymeric matrix. Exemplary synthetic polymers that can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, polyvinyl chloride, polystyrene and polyvinyl pyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. One preferred example of this type of polymer is the Matrix-Driven Delivery (MDD™) Pellet System of Innovative Research of America (Sarasota, Fla.), which contains in a matrix the active agent and carrier-binder excepients including cholesterol, lactose, celluloses, phosphates and stearates.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein by reference, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The invention includes the use of a sustained release implant to deliver ACAT inhibitors for the treatment of neurological disorders and atherosclerosis disorders as well as subjects at risk of developing such disorder. “Sustained” release, as used herein, means that the implant is configured to deliver therapeutic levels of the active ingredient for at least 7 days, and preferably at least 14 days, more preferably at least 30 days, still more preferably at least 2 months, yet more preferably at least 3 months, and most preferably at least 4 months. Longer term sustained release implants (e.g., that release active agent for at least 6 months) also are contemplated as providing greater convenience for patient and physician. At the end of this time period, another implant can be implanted for a similar length of time or a different length of time, depending on such factors as patient tolerance of the therapy, effectiveness of the therapy, and results of diagnostic testing to measure disease state (i.e., regression or progression of the disorder). Sustained release implants are well known to those of ordinary skill in the art and include some of the release systems described above.

Using the sustained release delivery system of the invention, a subject is treated for a length of time that generally will be at least one month, more preferably at least 2 months, still more preferably at least 4 months, 6 months, 8 months, 10 months, or more. Given the nature of ACAT-related diseases such as Alzheimer's disease and atherosclerosis, it is contemplated that the usual course of treatment will include administration of sustained release formulations of ACAT inhibitors for greater than 1 year, possibly for an indefinite period of time.

When implanted for the treatment of a ACAT-related neurological disorder, such as Alzheimer's disease, an implant or pump may be positioned at or near the area of the brain or nervous system affected by or involved in the neurological disorder. When implanted for the treatment of an ACAT-related atherosclerosis disorder, the implant pump may be positioned at or near the affected blood vessel(s) or organs.

Some embodiments of the invention include methods for treating a subject to ameliorate or prevent an ACAT-related disease or disorder (i.e., therapeutic treatment), which may be associated with abnormal levels of Aβ production. The methods involve administering to a subject who is known to have, is suspected of having, or is at risk of having an abnormal level of ACAT activity, an ACAT inhibitor for treating the disorder. Another aspect of the invention involves reducing the risk of an ACAT-related disease or disorder (i.e., prophylactic treatment), by the use of treatments and/or medications to modulate levels of ACAT activity.

An effective amount of an ACAT inhibitor is that amount effective to decrease ACAT activity in a subject. In the case of Alzheimer's disease, an effective amount may be an amount that inhibits (reduces) the levels of ACAT activity such that Aβ accumulation is reduced in the subject. For atherosclerosis, an effective amount may be an amount that inhibits (reduces) the levels of ACAT activity such that arterial plaque formation is reduced in the subject.

A response to a prophylatic and/or therapeutic treatment method of the invention can, for example, also be measured by determining the physiological effects of the treatment or medication, such as the decrease or lack of disease symptoms following administration of the treatment or pharmacological agent. Other assays will be known to one of ordinary skill in the art and can be employed for measuring the level of the response. For example, the behavioral and neurological diagnostic methods that are used to ascertain the likelihood that a subject has Alzheimer's disease, and to determine the putative stage of the disease can be used to ascertain the level of response to a prophylactic and/or treatment method of the invention. The amount of a treatment may be varied for example by increasing or decreasing the amount of a therapeutic composition, by changing the therapeutic composition administered, by changing the dosage or length of administration and so on. The effective amount will vary with the particular condition being treated, the age and physical condition of the subject being treated, the severity of the condition, the duration of the treatment, the nature of the concurrent therapy (if any), the specific route of administration, and the like factors within the knowledge and expertise of the health practitioner. For example, an effective amount can depend upon the degree to which an individual has abnormal levels of ACAT activity.

The factors involved in determining an effective amount are well known to those of ordinary skill in the art and can be addressed with no more than routine experimentation. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

The therapeutically effective amount of a pharmacological agent of the invention as used to treat an Aβ accumulation-associated disorder is that amount effective to inhibit or reduce Aβ production and/or accumulation, and thereby to treat, inhibit, prevent, or eliminate the Aβ accumulation-associated disorder. Therapeutically effective amounts of a pharmacological agent of the invention as used to treat an atherosclerosis disorder is that amount effective to inhibit or reduce atherosclerosis and/or plaque accumulation, and thereby to treat, inhibit, prevent, or eliminate the atherosclerosis disorder.

For example, testing can be performed to determine the levels of ACAT activity in a subject's tissue and/or cells. Additional tests useful for monitoring the onset, progression, and/or remission, of Aβ accumulation-associated disorders or atherosclerosis disorders such as those described above herein, are well known to those of ordinary skill in the art. As would be understood by one of ordinary skill, for some disorders (e.g., Alzheimer's disease and atherosclerosis) an effective amount would be the amount of a pharmacological agent of the invention that decreases the levels of ACAT activity to a level that ameliorates the disorder, as determined by the aforementioned tests.

In the case of treating a particular disease or condition the desired response is inhibiting the progression of the disease or condition, e.g., Alzheimer's disease or atherosclerosis. This may involve only slowing the progression of the disease temporarily, although more preferably, it involves halting the progression of the disease permanently. This can be monitored by routine diagnostic methods known to one of ordinary skill in the art for any particular disease. For diseases such as Alzheimer's disease, the load of amyloid beta-containing plaques in neuronal tissue can be determined by diagnostic imaging methods. Example of such methods include Bacskai et al., J Cereb Blood Flow Metab. 22(9):1035-41, 2002; Klunk et al., J Neuropathol Exp Neurol. 61(9):797-805, 2002; Mathis et al., J Med Chem. 46(13):2740-2754, 2003; Ono et al., Nucl Med Biol. 30(6):565-571, 2003; and Bacskai et al., Proc. Natl. Acad. Sci. USA 100(21): 12462-12467, 2003. For atherosclerosis, for example, the amount and/or size of atherosclerotic plaques can be measured using standard medical imaging techniques. The desired response to treatment of the disease or condition also can be delaying the onset or even preventing the onset of the disease or condition.

The pharmaceutical compositions used in the foregoing methods preferably are sterile and contain an effective amount of a pharmacological agent for producing the desired response in a unit of weight or volume suitable for administration to a patient.

The doses of pharmacological agents administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The dosage of a pharmacological agent of the invention may be adjusted by the individual physician or veterinarian, particularly in the event of any complication. A therapeutically effective amount typically varies from 0.01 μg/kg/day to about 1000 mg/kg/day, preferably from about 0.1 μg/kg/day to about 200 mg/kg/day, more preferably from about 1 μg/kg/day to about 20 mg/kg/day, and more preferably still from about 5 μg/kg/day to about 10 mg/kg/day.

Various modes of administration will be known to one of ordinary skill in the art which effectively deliver the pharmacological agents of the invention to a desired tissue, cell, or bodily fluid. The administration methods include a variety of sites for administration of sustained release implants, including subcutaneous, intracranial and intracavity. The choice of the site and mode of administration may be determined by the skilled artisan in accordance with the properties of the active agent (e.g., hydrophobicity, solubility, ability to cross the blood brain barrier, etc.) as well as the type of disease being treated and the state of the disease.

As used herein, a “sustained release implant” and similar terms denotes a pharmaceutical formulation that includes an active agent (e.g., ACAT inhibitor) contained within a non-active portion that does not dissolve or dissipate or release the active agent except in a sustained and preferably constant manner. Such sustained release implants have two advantages: the active agent is released with known release kinetics (preferably substantially constant release) and is released for an extended period of time, as compared to orally or parenterally administered formulations. Pump-based systems for sustained release provide similar benefits.

The invention is not limited by the particular modes of administration disclosed herein. Standard references in the art (e.g., Remington's Pharmaceutical Sciences, 18th edition, 1990) provide modes of administration and formulations for delivery of various sustained release pharmaceutical preparations and formulations including pharmaceutical substrates and carriers. Other protocols, sustained release formulations, and modes of administration which are useful for the administration of pharmacological agents of the invention will be known to one of ordinary skill in the art, in which the dose amount, schedule of administration, sites of administration, duration of administration, kinetics of administration and the like vary from those presented herein.

Administration of pharmacological agents of the invention to mammals other than humans, e.g., for testing purposes or veterinary therapeutic purposes, is carried out under substantially the same conditions as described above. Some of these conditions are exemplified in the Examples provided below. It will be understood by one of ordinary skill in the art that this invention is applicable to both human and animal diseases including Aβ accumulation-associated disorders of the invention. Thus, this invention is intended for use in husbandry and veterinary medicine as well as in human therapeutics.

When administered, the ACAT inhibitor compounds (also referred to herein as active agents, therapeutic compounds and/or pharmaceutical compounds) of the present invention are administered in pharmaceutically acceptable preparations. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts. Preferred components of the composition are described above in conjunction with the description of the pharmacological agents and/or compositions of the invention.

A pharmacological agent, composition or sustained release implant containing the agent may be combined, if desired, with a pharmaceutically acceptable carrier. The term “pharmaceutically acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the pharmacological agents of the invention, and with each other, in a manner such that there is no interaction which would substantially impair the desired pharmaceutical efficacy. The characteristics of the carrier will depend on the route of administration, the site of implantation, the duration of the implant, the chemical properties of the therapeutic, etc.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds. The pharmaceutical compositions also may contain, optionally, suitable preservatives, such as: benzalkonium chloride; chlorobutanol; parabens and thimerosal.

The invention will be more fully understood by reference to the following examples. These examples, however, are merely intended to illustrate the embodiments of the invention and are not to be construed to limit the scope of the invention.

### EXAMPLES

Methods

Animals and Drug Treatment

hAPP transgenic mice overexpress human APP₇₅₁ with the London (V717I) and Swedish (K670M/N671L) mutations under the regulatory control of the neuron specific murine (m)Thy-1 promoter (mThy-1-hAPP₇₅₁; heterozygous with respect to the transgene, on a C57BL/6 F3 background) (Rockenstein et al., 2001). The hAPP colony was sustained by crossing transgenic APP₇₅₁ with C57BL/6 (Harlan Winkelman, Germany). Corresponding littermates were used for control studies. All mice were housed according to standard animal care protocols, fed ad libitum with standard chow diet, and maintained in a pathogen-free environment in single ventilated cages at JSW Research. The transgenic status of each animal was confirmed by real time PCR of tail snips using specific primers and the appropriate hybridization probe. A modified Irvine test was regularly performed prior to the experiment to assess the neurological status of the animals; those showing disturbances were excluded from the experiments. Mice were then randomly assigned to different treatment groups and individually coded. Investigators performing behavioral testing, biochemical analyzes and histomorphological evaluations of the brains were blinded in terms of group allocation of the mice. CP-113,818, a potent inhibitor of both ACAT1 and ACAT2 (Chang et al., 2000), was graciously provided by Dr. James Harwood, Pfizer (Groton, Conn.). The drug was compounded in release pellets for continuous dosing by Innovative Research of America (Sarasota, Fla.). Only the test compound, but not carrier biopolymers and substances in the pellets, are released into the bloodstream. Pellets were generated to provide either 21 day or 60 days of continuous drug delivery. For implantation of pellets, animals were anesthized with isofluorance. Then, sterile pellets containing either CP-113,818 or placebo were implanted subcutaneously along the anterolateral aspect of the left shoulder with a special precision trocar in accordance with the suppliers instructions. There was no evident need for additional hemostasis, and no signs of infection, discomfort, or distress were observed in association with the implantation and treatment.

Tissue Sampling

Animals were sacrificed the day after the last MWM training, on day 57 of treatment. Blood was obtained by cardiac puncture and total serum was used for cholesterol determination. Transcardiac perfusion with 4C PBS was performed, followed by dissection. Brains were removed, divided along the sagittal plane and then either frozen in liquid nitrogen or imersion fixed with 4% paraformaldehyde for histologic evaluation. Liver was frozen in liquid nitrogen, and adrenal glands were fixed in 4% paraformaldehyde followed by standard paraffin embedding and sectioning. Hematoxylin and eosin stained 6-8 um sections were analyzed for evidence of CP-113,818 toxicity.

Cholesterol Determinations

Tissues were homogenized in the presence of trypsin (10 mg/ml) in a Dounce homogenizer on ice. Protein concentration of the homogenate was determined using the BCA protein assay kit (Pierce). The tissue homogenate was extracted in chloroform:methanol (2:1, v/v) overnight. Before drying the chloroform phase, polyoxyethylene-9-lauryl ether (Sigma; 5 μl/ml of extract) was added. Dried lipid pellets were dissolved in water and total amount of cholesterol was determined with an enzymatic assay (Sigma; cat. no. 402-20). As control, free cholesterol was determined with a different enzymatic assay (R-Biopharm GmbH, Darmstadt, Germany; cat. no. E0139050). The amount of cholesteryl-esters was calculated by subtracting free from total cholesterol. Finally, the values were normalized to protein concentration of the tissue homogenate and expressed in mg of cholesterol per g of protein.

Brain Plaque Load and Synaptophysin Analyses

For histomorphological analyses, 8 to 15 histological sections (10 μm) were prepared from one hemisphere of 12 animals (6 placebo, 6 CP-113,818; 3 male, 3 female per group). Number and surface area of amyloid plaques were evaluated in both cortex and hippocampus. 6E10 (Signet; 1:5000; monoclonal) as primary antibody, and Cy™3 fluorescent goat anti-mouse IgG as secondary antibody (Jackson Immuno Research) were used for Aβ staining. Sections of the same brains were also used for synaptophysin staining (Chemicon; 1:5000; monoclonal). Synaptophysin immunoreactive spots were counted by light microscopy in the hippocampal CA1 region. The estimation of immunohistochemical reactions was done by computer-assisted quantification (IMAGE PRO PLUS, Media Cybernetics).

Aβ₁₋₄₀ and Aβ₁₋₄₂ Determinations

For Aβ determinations, frozen hemispheres were first homogenized in TBS-buffer (5 ml) containing a protease inhibitor cocktail (Protease Inhibitor Cocktail Set I, Calbiochem) and then centrifuged at 75000 g for 1 hour, as described elsewhere (Kawarabayashi et al., 2001). Supernatants were saved for soluble Aβ analyses (see below). Pellets were resuspended and further homogenized in 70% formic acid (1 ml), followed by centrifugation at 75000 g for 1 hour. Formic acid-supernatants were neutralized with 1M TRIS (19 ml) and used for ELISA determination (insoluble Aβ). Aβ₁₋₄₀ and Aβ₁₋₄₂ were assayed using commercially available ELISA kits (The Genetics Company®, Switzerland). Measurements were performed at least in duplicate. Soluble Aβ ELISAs: secreted Aβ₁₋₄₀ and Aβ₁₋₄₂ levels in the TBS homogenate were analyzed by a sandwich ELISA system as described previously using the following antibodies: Aβ₄₀=MM27 33.1.1 capture, MM32 13.1.1 for detection; Aβ₄₂=MM27 33.1.1 capture, MM26 4.1.3 for detection (Suzuki et al., 1994).

Behavior

53 days after the treatment, mice were trained in the Morris water maze (MWM) spatial navigation task in which a mouse swims to find a hidden platform, using visual cues. The task is based on the principle that rodents are highly motivated to escape from a water environment by the quickest, most direct route. This task is carried out in a circular pool (1 meter diameter) filled with water (22±1° C.) and virtually divided into four quadrants. A platform is placed in the southwest quadrant 1 cm beneath the surface of the water. The computer randomly changes the starting positions of an animal. On four consecutive days mice attend three trials per day (each lasting for a maximum of 60 seconds). Escape latency (time to find the hidden platform), and length of swimming path were measured. Significant differences of the behavioral results were calculated with the Mann Whitney U-test.

Western Blot Analysis

For Western blot analyses, frozen hemispheres of non-transgenic littermates were homogenized in TBS buffer containing 5 mM EDTA, 1 mM 1,10-phenanthroline (ICN Biomedicals), 20 μM ALLN (BioMol) and a protease inhibitor mixture (Roche). Homogenates were centrifuged at 100000 g for 1 hour at +4° C. The supernatants were used for analyses of secreted APP forms and ApoE, while the pellets extracted with the homogenization buffer containing 1% Triton X-100 and 0.2% SDS. 120 μg of total protein were resolved on 4-12% or 12% NuPAGE Bis-Tris gels (Invitrogen) under reducing conditions, and Western blots were performed as described previously (Puglielli et al., 2001). For analysis of Notch-ICD (NICD), 250 μg of total protein was immunoprecipitated with an NICD-specific antibody (cleavage site (Val1744)-specific; Cell Signaling Technology) according to manufacturer's recommendations. Antibodies used for Western blots were R1736 (specific for sAPP-α; a gift from Dr. D. J. Selkoe, Brigham and Women's Hospital, Boston, Mass.), Abl4 (PS 1-NTF; a gift from Dr. S. E. Gandy, Thomas Jefferson University, Philadelphia, Pa.), Pen2 (a gift from Dr. S. S. Sisodia, University of Chicago, Chicago, Ill.), N-Cadherin (C32, C-terminal; BD Transduction Laboratories), ApoE (Santa Cruz Biotechnology), β-tubulin (Sigma), BACE1 and Nicastrin (Affinity BioReagents). Rbt×APP (CTF), 22C11 (APP-NTF) and Rbt×PSI (PS 1-CTF) were from Chemicon International. BACE stainings (N-terminal; Affinity BioReagents) were confirmed with two additional antibodies; Ms×BACE1 (Chemicon International) and BACE (C-terminal; Affinity BioReagents). Densitometric analyses were performed using the QuantityOne software package (Bio-Rad). Statistical significance was determined by Student's t test. For Western blot analyses shown in supplemental figures, neutralized formic acid fractions (approximately 100 μg of total protein) were precipitated by adding 50% trichloroacetic acid to 15.5% final concentration. After 30 min incubation and centrifugation at 4° C., the pellets were solubilized by adding 10 μl of 2M Tris base and 10 μl of 4×LDS-PAGE loading buffer (NuPage, Invitrogen). Before loading, the samples were incubated for 30 min at 37° C., freeze-thawed once and boiled. Western blot analysis was performed as described above.

In Vitro Fluorometric BACE1 Activity Assay

A fluorometric β-secretase activity kit was used for detecting BACE1 activity in vitro. Reaction was done according to the manufacturer's instructions with slight modifications. Briefly, 1 μg of purified recombinant human BACE1 (R&D systems) was resuspended in 10 μl of 1× cell extraction buffer (R&D systems) and incubated with EDANS/DABCYL-REEVNLDAEFKR substrate peptide in the absence or presence of indicated account of CP-113,818 for 2 hours. Released EDANS fluorescence was measured by a fluorescent plate reader (Analyst AD, LJL Biosystems). As a negative control, a specific β-secretase inhibitor, GL-189 (Calbiochem) was added to one of the samples with the indicated concentration.

In Vitro Generation of APP Intracellular Domain (AICD)

Membrane preparation and in vitro generation of AICD were performed as described (Gu et al., 2001; Kim et al., 2002). P2 membrane fractions were prepared from CHO cells overexpressing wild type APP and resuspended in Buffer H (20 mM HEPES, 150 mM NaCl, 10% glycerol, 5 mM EDTA, pH 7.4) with protease inhibitors. In vitro cleavage experiments were performed by incubating the membrane fractions at 0° C. or 37° C. for 1 hour with the indicated amounts of CP-113,818. As a negative control, a specific γ-secretase inhibitor, L-685,458, was added to one of the samples.

Turbidity Assay

Aβ₄₀ aggregation was monitored according to the method of Jarrett et al. (Jarrett et al., 1993). Briefly, Aβ₄₀ (100 μg/ml) was incubated at 37° C. in TBS in a 384-well plate (60 μl/well). Well turbidity was monitored for four days by following absorbance at 400 nm. Aβ₄₀ aggregation was promoted by the addition of “aged” (pre-incubated for 2 weeks at room temperature) Aβ₄₂ (Aβ₄₂/Aβ₄₀=1/10) (Jarrett et al., 1993) or Zn/histine buffer (50 μM Zn++in 350 μM histine) (Bush et al., 1994). Absorbance of test wells was blanked on signal from samples without Aβ₄₀. For some experiments, incubation buffers included CP-113,818 (10 μM).

Results

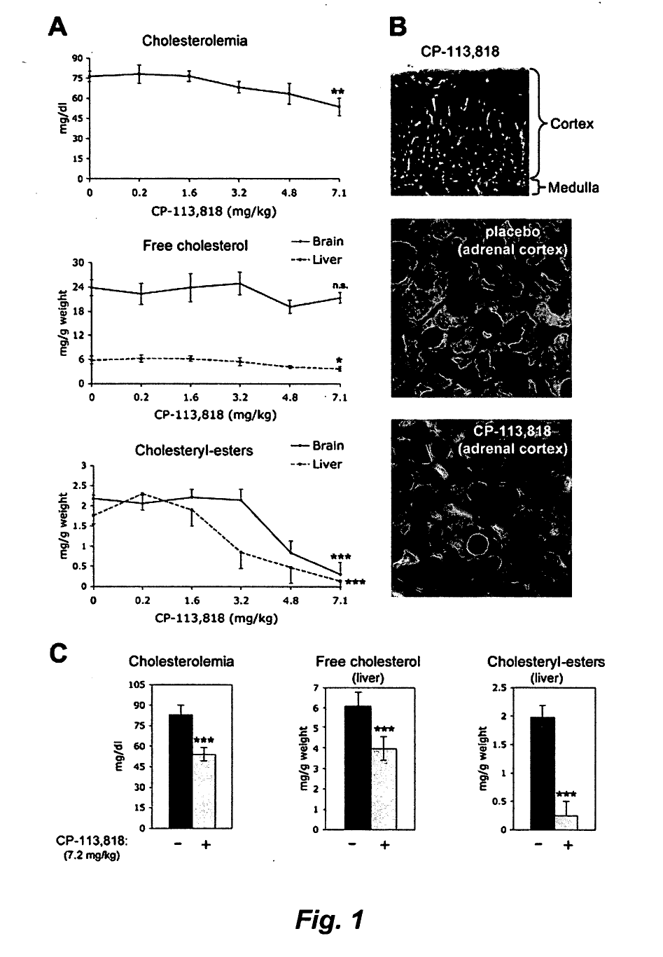

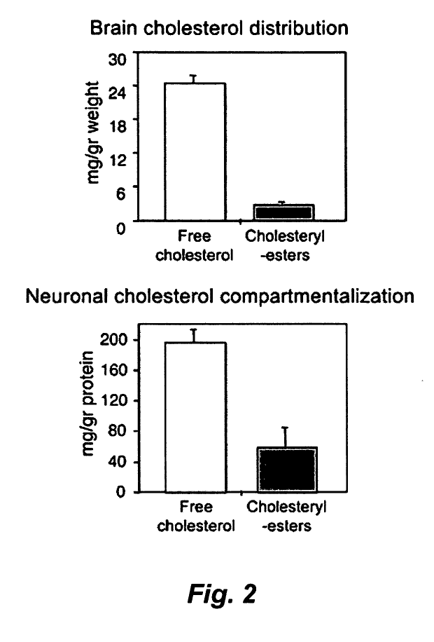

CP-113,818 is a fatty-acid anilide derivative designed to mimic the acyl-CoA substrate of ACAT and a potent ACAT inhibitor in vitro, cell culture, and animals (Chang et al., 2000; Marzetta et al., 1994; Puglielli et al., 2001). In order to determine the in vivo efficacy of CP-113,818 in reducing mouse brain cholesteryl-ester levels, we initially treated non-transgenic animals with the ACAT inhibitor. Most ACAT inhibitors are poorly absorbed when administered orally, and are quickly turned over in the blood or tissues (Marzetta et al., 1994). To minimize daily fluctuations in serum CP-113,818 levels in mice, we delivered the inhibitor via implantable slow-release biopolymer pellets. Pellets containing CP-113,818 were inserted surgically under dorsal skin to allow for continuous and controlled release of the active compound over an established period of time with effectively zero-order kinetics (Innovative Research of America). 18 non-transgenic mice at 3 months of age received 21 day-release biopolymer pellets containing 0, 0.2, 1.6, 3.2, 4.8, and 7.1 mg/kg per day of CP-113,818 (n=3 per dose; FIG. 1A). The highest dose used in these studies was far below the tolerated dose of 150 mg/kg per day for rats, even allowing for relatively low bioavailability in these animals (Marzetta et al., 1994). 7.1 mg/kg per day of CP-113,818 reduced total cholesterol levels by 29% (p<0.007) in the serum, in the absence of any evident effect on food consumption and body weight (FIG. 1A; data not shown). Hepatic free cholesterol and cholesteryl-esters were also decreased in a dose-dependent manner by up to 37% (p<0.03) and 93% (p<0.0004) respectively (FIG. 1A). As expected, CP-113,818 had no effect on free cholesterol levels in the brain, since free cholesterol is largely immobilized in myelin membranes and is not likely perturbed by ACAT activity. Initial levels of cholesteryl-esters, 2.2 mg/g of brain tissue, were similar to reported values of approximately 3.5 mg/g of rat hippocampal tissue (Champagne et al., 2003) and corresponded to ˜8% of total brain cholesterol. We found that free cholesterol:cholesteryl-ester ratios in mouse brains were approximately 10:1, but were only 3:1 in enriched neuronal primary cultures largely devoid of myelin (FIG. 2). 7.1 mg/kg of CP-113,818 inhibited cholesteryl-ester generation in mouse brains by 86% (p<0.0004; FIG. 1A). Thus, these data indicate that 7.1 mg/kg per day of CP-113,818 is effective in markedly reducing cholesteryl-ester levels in the brains of non-transgenic mice.

To characterize the effect of CP-113,818 treatment on AD-like pathology, we used hAPP mice that express human APP₇₅₁ containing the London (V717I) and Swedish (K670M/N671L) mutations under the regulatory control of the murine Thy-1 promoter (mThy1-hAPP₇₅₁) (Rockenstein et al., 2001). These animals develop detectable plaques in the neocortex and hippocampus at the ages of 4 and 6 months, respectively, and show memory deficits starting at 6 months of age. Given our prior observations that CP-113,818 reduced Aβ generation and not clearance, we used relatively young hAPP mice to minimize initial Aβ deposition. hAPP mice (n=12) at 4.5 months of age were administered 60-day release biopolymer pellets containing 13 mg of CP-113,818 (7.2 mg/kg per day), while age-matched animals were implanted with placebo pellets (n=12). Recent reports have shown that female mice have higher levels of Aβ and amyloid pathology than age-matched males in a variety of AD transgenic mouse models (Callahan et al., 2001; Lee et al., 2002; Wang et al., 2003), and that lovastatin may function in a gender-specific manner (Park et al., 2003). Therefore, we included similar numbers of male and female animals in both placebo and CP-113,818 groups to detect possible gender-specific effects of the inhibitor in our mouse model. Following 4 days of Morris water maze tests, the animals were sacrificed at day 57 to ensure continued presence of the inhibitor. At 7.2 mg/kg per day of CP-113,818 there was no histologic evidence of toxicity found in adrenal cortical cells, a potential class-related effect of ACAT inhibitors (FIG. 1B). Serum total cholesterol and liver free cholesterol/cholesteryl-esters were reduced to levels similar to those previously observed in non-transgenic animals (FIGS. 1A, C). Noteworthy, liver cholesteryl-ester levels decreased by 87% (p<0.0001) in hAPP mice treated for two months with CP-113,818 (FIG. 1C). Taken together, these experiments demonstrated that two months of CP-113,818 treatment effectively reduced cholesteryl-ester levels of hAPP mice in the absence of adrenal toxicity. The tissue requirements for assessment of Aβ burden in the brains of these animals precluded direct examination of their brain cholesteryl-ester levels.

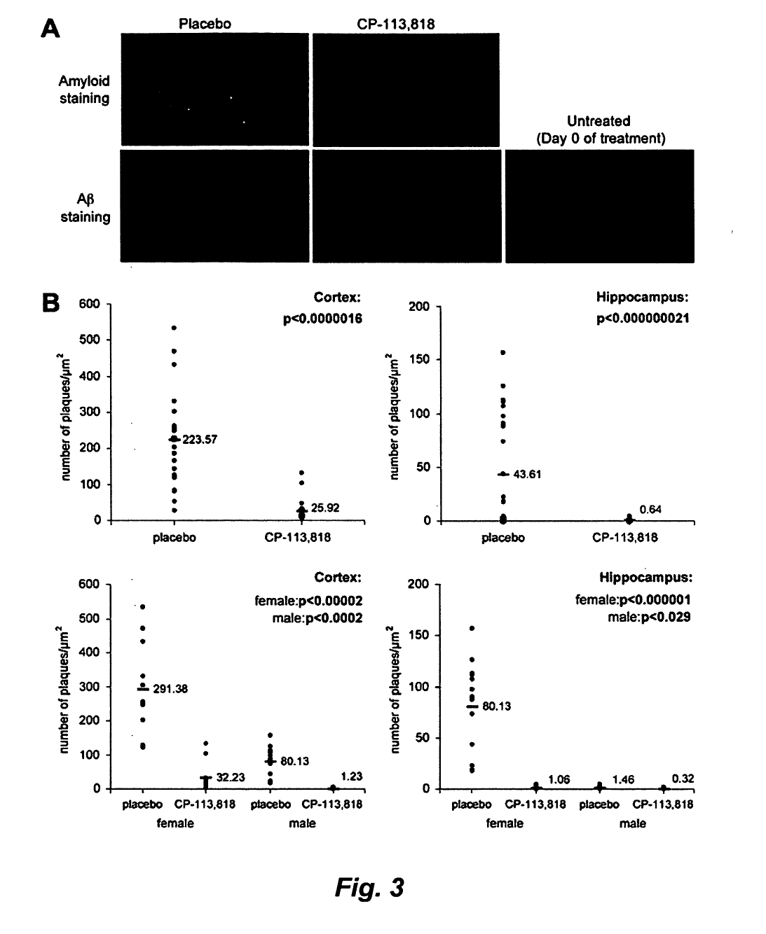

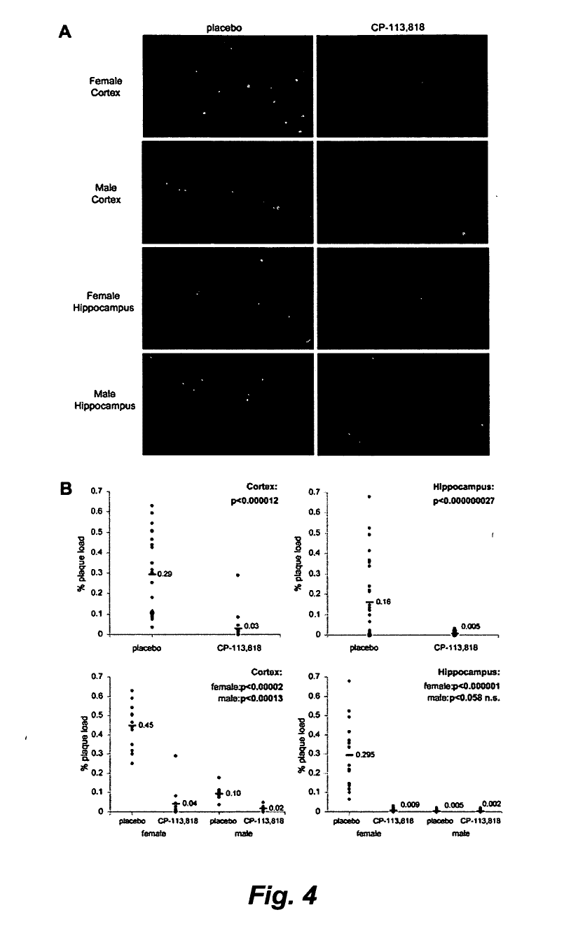

Brain plaque load was evaluated by thioflavin S and immunohistochemical stainings (FIG. 3). Thioflavin S-positive plaques were barely detectable in the cortex of hAPP mice at 6.5 months of age and absent in CP-113,818-treated animals (FIG. 2A). However, Aβ staining with the monoclonal antibody 6E10 revealed abundant Aβ deposits in placebo-treated mouse brains (FIGS. 3A, B). Individual differences in amyloid load reflected the well-known variability in Aβ accumulation that has been previously observed in transgenic mice, specifically at early stages of pathology (Johnson-Wood et al., 1997; Kawarabayashi et al., 2001). Amyloid load in the cortex of female animals was two-fold higher than in males, in agreement with previous studies in other mouse models of AD (Callahan et al., 2001; Lee et al., 2002; Wang et al., 2003). The hippocampus, slower than the cortex in developing amyloid pathology in hAPP mice, contained significant numbers of Aβ deposits only in female, but not in male, animals (FIG. 3B and FIG. 4). In the cortices of all animals tested, CP-113,818 treatment reduced plaque numbers by 88%, p<0.0000016. Importantly, when the two genders were considered separately, plaque numbers were reduced to an equal extent in females and males, by 89%, p<0.00002, and 88%, p<0.0002, respectively (FIG. 3B). Thus, CP-113,818 treatment decreased amyloid load in a gender-independent manner. Plaque burden, as indicated by the percent of cortex covered by plaques, was also markedly decreased in all animals (by 90%, p<0.000012). In females the decrease was from 0.45 to 0.04 percent plaque load (p<0.00002), while in males it was from 0.10 to 0.02 (p<0.00013; FIG. 4B). As mentioned above, amyloid load in the hippocampus of male animals was too low at this age for reliable assessment of changes caused by CP-113,818. However, the most dramatic effects of the ACAT inhibitor were observed in the hippocampus of female mice, where plaque number decreased by 99% (p<0.000001) and plaque coverage by 97% (p<0.000001) from 0.295 to 0.09 percent plaque load (FIG. 3B and FIG. 4B). Plaque numbers in the cortex of female animals were similar at the beginning and the end of CP-113,818 treatment (FIG. 3A), suggesting that the ACAT inhibitor prevents accumulation of newly formed plaques. Overall, these data show that CP-113,818 treatment was highly effective in reducing accumulation of amyloid plaques in hAPP mice and this effect was gender-independent.

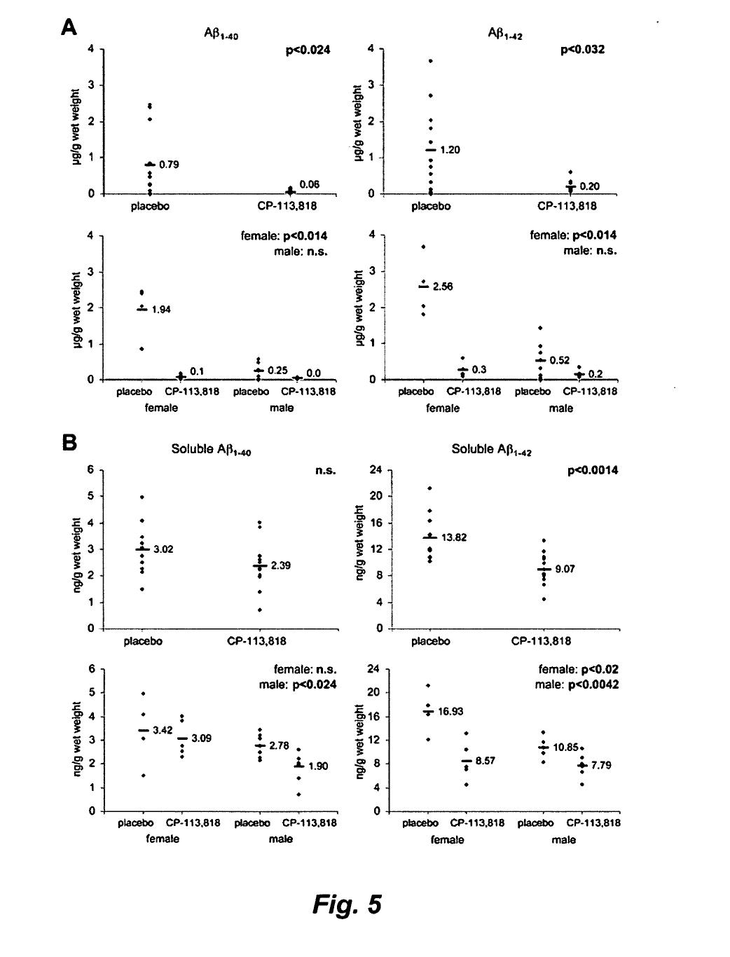

We next used a sandwich enzyme-linked immunosorbent assay (ELISA) to measure Aβ₁₋₄₀ and Aβ₁₋₄₂ levels in 70% formic acid extracts from brain homogenates (“insoluble” Aβ) and in the initial TBS fraction of the same homogenates (soluble Aβ). Similar to plaque load, Aβ levels varied widely, as expected in transgenic mice specifically at early stages of pathology (Johnson-Wood et al., 1997; Kawarabayashi et al., 2001). Treatment with CP-113,818 decreased Aβ₁₋₄₀ and Aβ₁₋₄₂ in the formic acid fraction to almost undetectable concentrations. An overall 92% (p<0.024) decrease in “insoluble” Aβ₁₋₄₀ as well as 83% (p<0.032) in “insoluble” Aβ₁₋₄₂ was detected in all animals after treatment with CP-113,818, with no significant difference in the Aβ₁₋₄₂/Aβ₁₋₄₀ ratio (FIG. 5A). Since brain extracts were obtained from entire hemispheres including cortices and hippocampi, the difference between male and female Aβ levels at this age was accentuated by the slowly developing amyloid pathology in male hippocampi (see FIG. 3B). When analyzed according to gender, only female “insoluble” Aβ was sufficiently elevated to obtain a highly significant decrease by CP-113,818 treatment, with Aβ₁₋₄₀ down by 96% (p<0.014) and Aβ₁₋₄₂ down by 90% (p<0.014; FIG. 5A). Importantly, soluble Aβ levels were also decreased in the initial TBS fraction, with Aβ₁₋₄₂ showing a significant 34% reduction (p<0.0014). This decrease was statistically significant in both genders. Soluble Aβ₁₋₄₀ levels were close to the limit of detection, but a trend toward decrease was observed (20%) in all animals, significant only in males (FIG. 5B). Thus, three different methods of detection, thioflavin S and Aβ stainings and Aβ ELISAs, independently showed that CP-113,818 treatment is highly effective in reducing amyloid pathology in hAPP mice. The decrease in soluble Aβ suggests that CP-113,818 may either reduce Aβ generation or accelerate its catabolism or clearance.

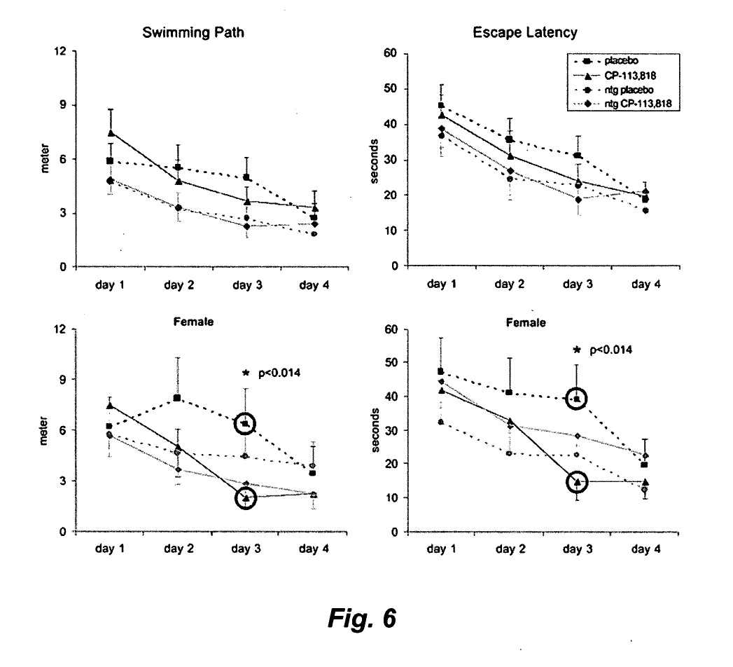

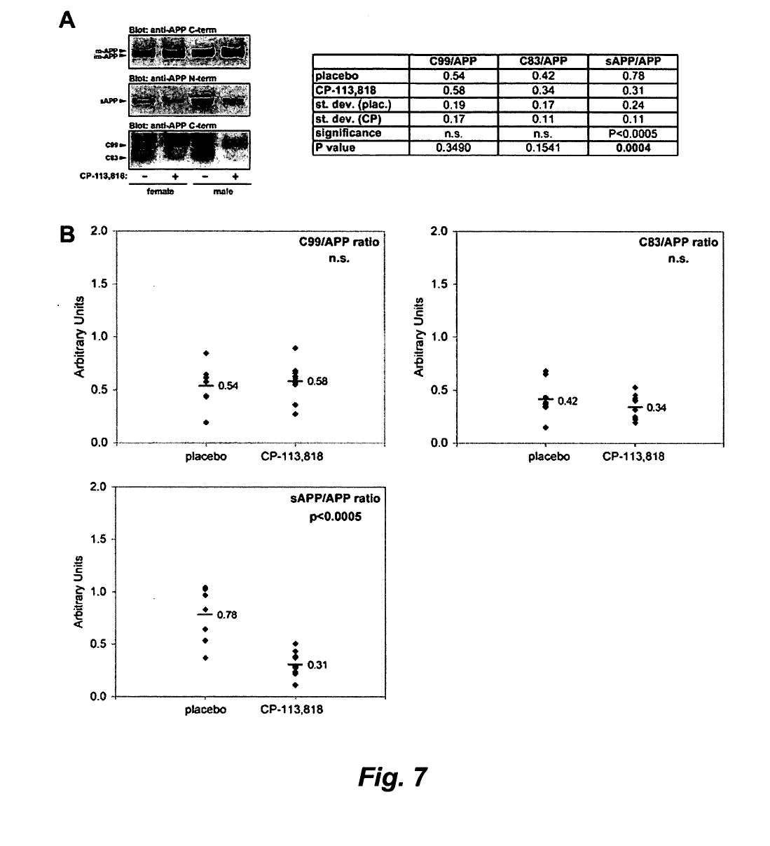

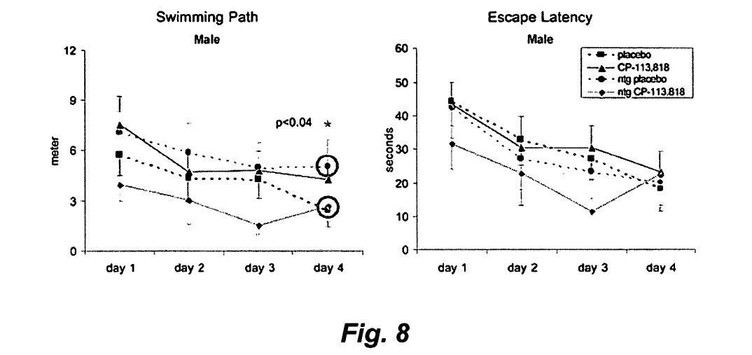

To analyze the effect of CP-113,818 treatment on cognitive function of hAPP mice, we performed Morris water maze spatial learning and memory tests on the animals. Since hAPP mice develop cognitive deficits starting at 6 months of age, the 6.5 month-old animals in our study were only expected to show slight deterioration due to Aβ accumulation, when compared to non-transgenic littermates. Consistently, length of swimming path and time latency required for finding the submerged platform revealed only a slight disturbance in spatial learning and memory in transgenic animals, compared to their littermates. Clear impairment of cognitive function due to Aβ accumulation was only observed in female transgenic mice. In these animals, CP-113,818 treatment reversed the observed impairment in spatial learning (p<0.014; FIG. 6). Compared to placebo, CP-113,818 treatment resulted in a significant improvement of learning between day 1 and day 3 (p<0.016). CP-113,818 treatment did not affect spatial performance of non-transgenic female mice, consistent with lack of Aβ deposition in these animals. Only on day four, CP-113,818-treated male non-transgenic animals performed better than the placebo-treated cohort regarding swimming path (p<0.04; FIG. 7), but not escape latency; however, the improvement between day 1 and day 3 or 4 was identical for both groups. All hAPP mice taken together showed a trend toward acquiring the task quickly and reaching a plateau of performance on day three when treated with CP-113,818 (p<0.07; FIG. 6). This could have been due to a ceiling effect owing to maximum swimming speed. Compared to placebo, the improvement between day 1 and day 3 due to CP-113,818 treatment was significant (p<0.033). As expected, CP-113,818 did not induce significant changes in synaptic density as assessed by synaptophysin immunoreactivity, consistent with lack of differences between non-transgenic versus transgenic animals given their young age (FIG. 8). Although testing of more mice at an older age will be needed to further assess the effect of decreased amyloid pathology on spatial learning and memory, improved performance in a Morris water maze test correlated well with the highly significant decrease of overall plaque load and Aβ in CP-113,818-treated female animals.

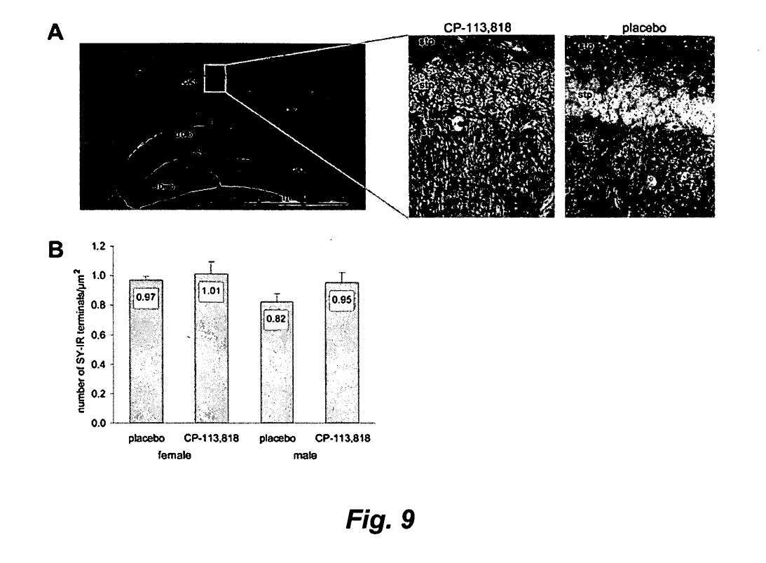

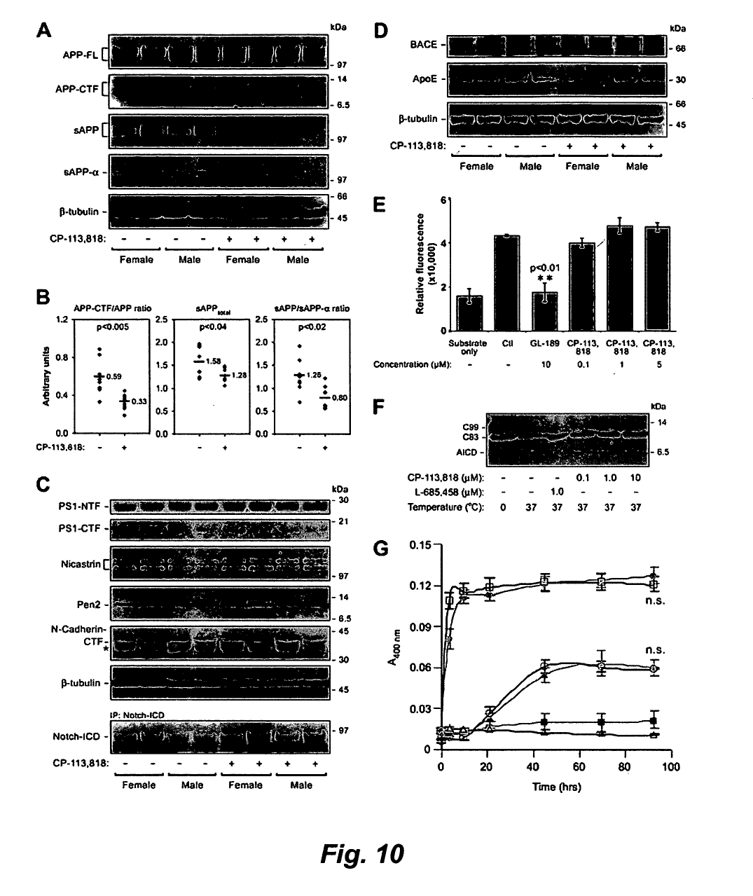

Our previous cell-based studies have shown that ACAT inhibition reduces Aβ generation and alters APP processing by all three secretases (Puglielli et al., 2001). To determine whether CP-113,818 treatment modified brain APP processing in hAPP mice, we initially resolved formic acid-extracted brain proteins by SDS-PAGE to detect APP and its proteolytic derivatives. Western blot analysis showed that formic acid-extracted full-length APP and APP C-terminal fragments (CTFs), C83 and C99, were unchanged by CP-113,818. However, levels of the secreted cleavage products of both α- and β-secretases (sAPP) were reduced by treatment with the ACAT inhibitor albeit not nearly as dramatically as Aβ (FIG. 9). Inhibition of sAPP production in the absence of a change in CTFs was somewhat surprising, and possibly an artifact caused by formic acid extraction. Because tissue from treated hAPP mice had been extracted with formic acid or fixed for histological studies, we looked at levels of APP-CTFs in total brain lysates from the non-transgenic littermates used in the Morris water maze tests. Using non-transgenic animals for these experiments also afforded an assessment of the effects of CP-113,818 on endogenous APP processing. CP-113,818 reduced liver cholesterol/cholesteryl-esters and serum cholesterol of non-transgenic littermates similarly to the transgenic animals shown in FIG. 1C (data not shown). Endogenous mouse APP-CTF levels in brain lysates from CP-113,818-treated animals decreased by 44% when compared to placebo-treated mice (n=8, p<0.005, values normalized to APP holoprotein levels; FIG. 10A, B). We could not resolve C99 and C83 from these samples due to smearing in the low molecular weight range because of high protein load (FIG. 10A). However, we were able to specifically detect secreted APPα (sAPPα), the N-terminal product of α-secretase cleavage, in the soluble TBS fraction of the initial homogenate. sAPPα showed a trend toward decreasing (3.4%, p<0.14=n.s.) in CP-113,818-treated animals, while total sAPP, which also includes the β-secretase product sAPPβ, was significantly reduced by 19.1% (p<0.038, FIG. 10A,B). A 36% decrease (p<0.013) in the ratio between total sAPP and sAPPα indicates that inhibition of α-secretase in ACAT inhibitor-treated mice is not as pronounced as that of other ectodomain-shedding proteases (e.g BACE). We next asked whether γ-secretase component levels and γ-activity were affected by CP-113,818 in the non-transgenic brain lysates. We did not detect significant changes in levels of PS1 N- and C-terminal fragments, nicastrin, and pen-2 (FIG. 10C; 8 of 16 samples shown, as in all other figures). Nicastrin maturation was also unaffected by CP-113,818 treatment. To detect potential changes in γ- and α-secretase-like activities for substrates other than APP, we tested two γ-secretase substrates, Notch and N-cadherin (De Strooper et al., 1999; Marambaud et al., 2003). Both proteins are also known to undergo an α-secretase-like cleavages, mediated by TACE for Notch and a metallo-protease for N-cadherin (Brou et al., 2000; Marambaud et al., 2003). Notch intracellular domain (NICD) represents the final γ-secretase cleavage product, and its levels are indicative of changes in both α-secretase-like and γ-secretase activities for Notch. NICD levels were statistically unchanged by CP-113,818 treatment, when the protein was immunoprecipitated from non-transgenic mouse brain lysates (FIG. 10C). Similarly, levels of N-cadherin-CTF1, the product of metallo-protease cleavage and a substrate for γ-secretase, were unaffected by the ACAT inhibitor (FIG. 10C). These data indicate that processing of at least two γ-secretase substrates was not altered by CP-113,818, and that α-secretase-like cleavages of these substrates were also unaffected. BACE protein levels were also unchanged in brain lysates of CP-113,818-treated animals (FIG. 10D). One mechanism that would explain our data is that ACAT inhibition may result in an interaction between APP and an unknown protein, blocking access of the three secretases to APP instead of direct inhibition of each secretase. Expression of ApoE, an essential protein in both brain lipid metabolism and Aβ deposition/clearance, was also unchanged by ACAT inhibition (FIG. 10D). In an effort to exclude potential direct effects of CP-113,818 on β- and γ-secretase cleavages of APP in the absence of changes in cholesterol metabolism, we performed in vitro β- and γ-secretase assays employing purified recombinant BACE1 and membrane fractions isolated from Chinese hamster ovary (CHO) cells harboring endogenous γ-secretase, respectively. Both BACE1 and γ-secretase activities were unaffected by increasing amounts of CP-113,818 in the reaction mixture (FIG. 10E,F). Finally, we tested whether CP-113,818 directly modulates in vitro aggregation of Aβ₄₀ promoted by zinc or Aβ₄₂ (Bush et al., 1994; Jarrett et al., 1993). CP-113,818 failed to directly inhibit aggregation of Aβ₄₀ in vitro, when promoted by zinc (p<0.56) or Aβ₄₂ (p<0.96) (FIG. 10G). In the absence of zinc or Aβ₄₂, Aβ₄₀ alone did not significantly aggregate over 4 days (p<0.671). Together, these data show that APP CTFs, total sAPP, and, to a lesser extent, sAPPα levels are decreased in the brains of CP-113,818-treated non-transgenic mice in the absence of any apparent change in the levels or processing of all control proteins tested. Although these data, together with decreased soluble Aβ in hAPP mice, argue strongly that the ACAT inhibitor reduces Aβ generation, they do not exclude additional beneficial effects of altered cholesterol metabolism on Aβ aggregation, catabolism, and/or clearance.

Current treatment approaches for AD based on lowering Aβ levels can be distinguished based on their respective targets (Citron, 2002). BACE1 and γ-secretase are the only targets that directly modulate Aβ generation, while other strategies target Aβ aggregation (e.g., small-molecule inhibitors, metal chelators) and/or clearance (AP immunotherapy). ACAT inhibitors, along with statins and non-steroidal anti-inflammatory drugs (NSAIDs), fit into a third class of a more generic treatment approach, based on targets that indirectly modulate Aβ generation. This class of treatment strategy provides additional benefits to reducing Aβ production. For instance, statins offer the added value of vascular protection and reduced cholesterolemia, while NSAIDs lower chronic brain inflammation (Citron, 2002; Cucchiara and Kasner, 2001). Similarly, ACAT inhibitors provide lipid-lowering benefits while being highly effective in inhibiting AD generation in our AD mouse model. Although CP-113,818 is not commercially available, avasimibe, a well-studied ACAT inhibitor produced by Pfizer (Groton, Conn.), has reached phase III trials for vascular disease and atherosclerosis. An experiment published in a published patent application (Bisgaier C. L. and Emmerling M. R., WO 99/38498) shows that 4 μM avasimibe, also called CI-1011, inhibits Aβ generation from CHO cells by approximately 60%. Our results suggest that slow-release biopolymer administration of ACAT inhibitors such as avasimibe and CP-113,818 at low doses, may be considered as a novel strategy for the treatment and prevention of Alzheimer's disease and other ACAT-related disorders.

Here we have identified a highly effective anti-amyloid treatment in a transgenic mouse model of AD, acting via inhibition of the cholesterol-esterifying enzyme ACAT. This treatment was gender-independent in our mouse model. Current anti-amyloid treatment approaches for AD can be distinguished based on their respective targets (Citron, 2002). BACE1 and γ-secretase are the only targets that directly modulate Aβ generation, while other strategies target Aβ aggregation (e.g. small-molecule inhibitors, metal binders) and/or clearance (e.g. Aβ immunotherapy). ACAT inhibitors, along with statins, would now fit into a third class of compounds based on targets that indirectly modulate Aβ generation. ACAT inhibition provides additional lipid-lowering benefits while being highly effective in inhibiting formation of Aβ pathology in the hAPP transgenic model of amyloid deposition.

Our previous cell-based studies indicate that ACAT inhibition reduces cholesteryl-ester production, which results in decreased Aβ generation (Puglielli et al., 2001). In those studies two different ACAT inhibitors, CP-113,818 and Dup128, lowered cholesteryl-ester production by up to 45% (Puglielli et al., 2001). Our current study reveals a more robust decrease in mouse brain cholesteryl-ester levels, perhaps attributable to the slow-release biopolymer carrier which ensured continuous delivery of the inhibitor. While it is difficult to predict final concentrations of CP-113,818 in the brain, crossing of the blood brain barrier is suggested by the structure of this ACAT inhibitor, a small fatty acid analogue, and the marked reduction in brain cholesteryl-esters relative to serum cholesterol, by 86% and 29% respectively. A previous study had reported 100-fold fluctuations of CP-113,818 blood levels in monkeys after oral administration of the compound (Marzetta et al., 1994). Our own attempts to quantitate liver or brain CP-113,818 have failed, consistent with fast turn-over or clearance of the inhibitor (Marzetta et al., 1994). Although neuronal Aβ generation could be affected by serum cholesterol, as suggested by a number of studies correlating serum cholesterol with brain Aβ levels (Puglielli et al., 2003), it is unlikely that a 29% decrease in serum cholesterol would reduce brain Aβ to the levels found in the current study. A more likely explanation is that the 86% decrease in brain cholesteryl-esters is responsible for the observed reduction in Aβ accumulation in CP-113,818-treated animals.

In transgenic mice, soluble Aβ₄₂ only decreased by 34%, while “insoluble” Aβ by 83-99%. These data may be explained by the complex metabolism of these peptides in the brain, such that the net amount of soluble Aβ is the result of its rate of generation, but also deposition, degradation, and clearance (Saido, 1998). However, reduction in Aβ generation could largely account for the surprisingly robust effect of CP-113,818 on “insoluble” Aβ and amyloid plaques in mouse brains. Small changes in Aβ generation, exemplified by a 50% increase in APP gene dosage in Down syndrome patients, or catabolism may disproportionately accelerate Aβ pathology and age of onset of AD (Saido, 1998). Similarly, FAD mutations in the presenilin genes only increase Aβ₄₂ generation by approximately 1.2-3 fold, and yet cause early-onset forms of the disease that can strike from the third decade onward (Borchelt et al., 1996; Citron et al., 1997; Duff et al., 1996; Holcomb et al., 1998). Although it is tempting to attribute decreased Aβ pathology in CP-113,818-treated mice to reduced Aβ production, we cannot exclude that altered brain cholesterol metabolism may also affect Aβ aggregation, catabolism, and/or clearance in addition to APP processing. Further studies will be necessary to evaluate these aspects of our findings.

Direct comparison between the efficacy of CP-113,818 and statins (or statin-like compounds) is nearly impossible due to differences in animal models, methods of drug administration, and length of treatment employed in each study. Refolo et al. (Refolo et al., 2001) and Petanceska et al. (Petanceska et al., 2002) employed a double transgenic mouse line, PSAPP, in which Aβ deposition begins at 12 weeks of age (Holcomb et al.). 250 mg/kg/day of the cholesterol-lowering drug BM15.766 (Refolo et al., 2001) or 30 mg/kg/day atorvastatin (Petanceska et al., 2002) were administered orally starting at 8 weeks of age and ending after 5 and 8 weeks, respectively. BM15.766 reduced amyloid load by 53% and formic acid-ektracted Aβ₄₀ by 58% and Aβ₄₂ by 48% (Refolo et al., 2001). Atorvastatin lowered amyloid load by ˜62% (2.7-fold), with formic acid-extracted Aβ₄₀ and Aβ₄₂ decreasing by ˜60 (2.5-fold) and ˜50% (2-fold), respectively (Petanceska et al., 2002). In another study conducted in guinea pigs, ˜250 mg/kg/day of orally administered simvastatin reduced endogenous detergent-extracted total Aβ by ˜45% in 3 weeks of treatment (Fassbender et al., 2001). Interestingly, one report has shown that lovastatin treatment of 12 month-old Tg2576 mice (expressing FAD mutant APP) for 3 weeks did not affect amyloid load or brain Aβ levels in males while increasing Aβ pathology in female animals (Park et al., 2003). It is not clear whether this contrasting result reflects differences in genetic backgrounds, or the short treatment time beginning at a late age, after development of large Aβ deposits. Female Tg2576 mice have been reported to develop amyloid pathology before males, perhaps due to higher synaptic zinc content in female brains by the age of 12 months (Callahan et al., 2001; Lee et al., 2002). Although difficult to compare in efficacy to CP-113,818, it is clear that statins or statin-like drugs strongly reduce Aβ pathology in different animal models when administered prior to Aβ deposition. It can be speculated that the influence of ACAT inhibition on APP metabolism is additive to effects resulting from changes in total cholesterol levels. Thus, in principle statins and ACAT inhibitors together could exhibit synergy in positively impacting AD pathology in patients affected by the disease.

CP-113,818 has never been tested in clinical trials, while CI-1011 (avasimibe), a well-studied ACAT inhibitor produced by Pfizer, previously entered phase III trials for vascular disease and atherosclerosis. Avasimibe is considered safe for human use, with a good therapeutic window. Our results suggest that slow-release biopolymer administration of ACAT inhibitors may be considered as a potential strategy for the treatment and prevention of Alzheimer's disease, alone or in combination with statins.

### REFERENCES

Aguzzi, A., and Haass, C. (2003). Games played by rogue proteins in prion disorders and Alzheimer's disease. Science 302, 814-818.
Boerwinkle, E., Visvikis, S., Welsh, D., Steinmetz, J., Hanash, S. M., and Sing, C. F. 1987. The use of measured genotype information in the analysis of quantitative phenotypes in man. II. The role of the apolipoprotein E polymorphism in determining levels, variability, and covariability of cholesterol, betalipoprotein, and triglycerides in a sample of unrelated individuals. Am J Med Genet 27, 567-582.
Borchelt, D. R., Thinakaran, G., Eckman, C. B., Lee, M. K., Davenport, F., Ratovitsky, T., Prada, C. M., Kim, G., Seekins, S., Yager, D., et al. (1996). Familial Alzheimer's disease-linked presenilin 1 variants elevate Aβ1-42/1-40 ratio in vitro and in vivo. Neuron 17, 1005-1013.
Brou, C., Logeat, F., Gupta, N., Bessia, C., LeBail, O., Doedens, J. R., Cumano, A., Roux, P., Black, R. A., and Israel, A. (2000). A novel proteolytic cleavage involved in Notch signaling: the role of the disintegrin-metalloprotease TACE. Mol Cell 5, 207-216.
Burns, M., and Duff, K. (2002). Cholesterol in Alzheimer's disease and tauopathy. Ann N Y Acad Sci 977, 367-375.
Bush, A. I., Pettingell, W. H., Multhaup, G., d Paradis, M., Vonsattel, J. P. G. J. F., Beyreuther, K., Masters, C. L., and Tanzi, R. E. (1994). Rapid induction of Alzheimer Aβ amyloid formation by zinc. Science 265, 1464-1467.
Callahan, M. J., Lipinski, W. J., Bian, F., Durham, R. A., Pack, A., and Walker, L. C. (2001). Augmented senile plaque load in aged female beta-amyloid precursor protein-transgenic mice. Am J Pathol 158, 1173-1177.
Champagne, D., Pearson, D., Dea, D., Rochford, J., and Poirier, J. (2003). The cholesterol-lowering drug Probucol increases apolipoprotein e production in the hippocampus of aged rats: implications for Alzheimer's disease. Neuroscience 121, 99-110.
Chang, C. C., Sakashita, N., Omvold, K., Lee, O., Chang, E. T., Dong, R., Lin, S., Lee, C. Y., Strom, S. C., Kashyap, R., et al. (2000). Immunological Quantitation and Localization of ACAT-1 and ACAT-2 in Human Liver and Small Intestine. J Biol Chem 275, 28083-28092.
Chang, T. Y., Chang, C. C., Lu, X., and Lin, S. (2001). Catalysis of ACAT may be completed within the plane of the membrane. A working hypothesis. J Lipid Res 42, 1933-1938.
Citron, M. (2002). Alzheimer's disease: treatments in discovery and development. Nat Neurosci 5 Suppl, 1055-1057.
Citron, M. (2004). Beta-secretase inhibition for the treatment of Alzheimer's disease—promise and challenge. Trends Pharmacol Sci 25, 92-97.
Citron, M., Westaway, D., Xia, W., Carlson, G., Diehl, T., Levesque, G., Johnson-Wood, K., Lee, M., Seubert, P., Davis, A., et al. (1997). Mutant presenilins of Alzheimer's disease increase production of 42-residue amyloid β-protein in both transfected cells and transgenic mice. Nat Med 3, 67-72.
Corder, E. H., Saunders, A. M., Strittmatter, W. J., Schmechel, D. E., Gaskell, P. C., Small, G. W., Roses, A. D., Haines, J. L., and Pericak-Vance, M. A. 1993. Gene dose of apolipoprotein E type 4 allele and the risk of Alzheimer's disease in late onset families. Science 261, 921-923.
Cucchiara, B., and S. E. Kasner. 2001. Use of statins in CNS disorders. J Neurol Sci. 187:81-89.
De Strooper, B. (2003). Aph-1, Pen-2, and Nicastrin with Presenilin generate an active gamma-Secretase complex. Neuron 38, 9-12.
De Strooper, B., Annaert, W., Cupers, P., Saftig, P., Craessaerts, K., Mumm, J. S., Schroeter, E. H., Schrijvers, V., Wolfe, M. S., Ray, W. J., et al. (1999). A presenilin-1-dependent gamma-secretase-like protease mediates release of Notch intracellular domain Nature 398, 518-522.
Duff, K., Eckman, C., Zehr, C., Yu, X., Prada, C. M., Perez-tur, J., Hutton, M., Buee, L., Harigaya, Y., Yager, D., et al. (1996). Increased amyloid-β42(43) in brains of mice expressing mutant presenilin 1. Nature 383, 710-713.
Ehnholm, C., Lukka, M., Kuusi, T., Nikkila, E., and Utermann, G. 1986. Apolipoprotein E polymorphism in the Finnish population: gene frequencies and relation to lipoprotein concentrations. J Lipid Res 27, 227-235.
Fassbender, K., Simons, M., Bergmann, C., Stroick, M., Lutjohann, D., Keller, P., Runz, H., Kuhl, S., Bertsch, T., von Bergmann, K., et al. (2001). Simvastatin strongly reduces levels of Alzheimer's disease beta—amyloid peptides Abeta 42 and Abeta 40 in vitro and in vivo. Proc Natl Acad Sci USA 98, 5856-5861.
Frears, E. R., Stephens, D. J., Walters, C. E., Davies, H., and Austen, B. M. 1999. The role of cholesterol in the biosynthesis of beta-amyloid. Neuroreport 10, 1699-1705.
Glenner, G. G., and Wong, C. W. (1984). Alzheimer's disease: initial report of the purification and characterization of a novel cerebrovascular amyloid protein. BiochemBiophysResCommun 120, 885-890.
Gu, Y., Misonou, H., Sato, T., Dohmae, N., Takio, K., and Ihara, Y. (2001). Distinct intramembrane cleavage of the beta-amyloid precursor protein family resembling gamma-secretase-like cleavage of Notch. J Biol Chem 276, 35235-35238.
Hartmann, T. (2001). Cholesterol, A beta and Alzheimer's disease. Trends Neurosci 24, S45-48.
Holcomb, L., Gordon, M. N., McGowan, E., Yu, X., Benkovic, S., Jantzen, P., Wright, K., Saad, I., Mueller, R., Morgan, D., et al. (1998). Accelerated Alzheimer-type phenotype in transgenic mice carrying both mutant amyloid precursor protein and presenilin 1 transgenes. Nat Med 4, 97-100.
Jarrett, J. T., Berger, E. P., and Lansbury P. T, Jr. (1993). The carboxy terminus of the β amyloid protein is critical for the seeding of amyloid formation: implications for the pathogenesis of Alzheimer's disease. Biochemistry 32, 4693-4697.
Jarvik, G. P., Wijsman, E. M., Kukull, W. A., Schellenberg, G. D., Yu, C., and Larson, E. B. 1995. Interaction of apolipoprotein E genotype, total cholesterol level, and sex in prediction of Alzheimer disease in a case-control study. Neurology 45, 1092-1096.
Jick, H., G. L. Zomberg, S. S. Jick, S. Seshadri, and D. A. Drachman. 2000. Statins and the risk of dementia. Lancet. 356:1627-31.
Johnson-Wood, K., Lee, M., Motter, R., Hu, K., Gordon, G., Barbour, R., Khan, K., Gordon, M., Tan, H., Games, D., et al. (1997). Amyloid precursor protein processing and A beta42 deposition in a transgenic mouse model of Alzheimer disease. Proc Natl Acad Sci USA 94, 1550-1555.
Kang, J., Lemaire, H. G., Unterbeck, A., Salbaum, J. M., Masters, C. L., Grzeschik, K. H., Multhaup, G., Beyreuther, K., and Muller-Hill, B. (1987). The precursor of Alzheimer's disease amyloid A4 protein resembles a cell-surface receptor. Nature 325, 733-736.
Kawarabayashi, T., Younkin, L. H., Saido, T. C., Shoji, M., Ashe, K. H., and Younkin, S. G. (2001). Age-dependent changes in brain, CSF, and plasma amyloid (beta) protein in the Tg2576 transgenic mouse model of Alzheimer's disease. J Neurosci 21, 372-381.
Kim, D. Y., Ingano, L. A., and Kovacs, D. M. (2002). Nectin-1alpha, an immunoglobulin-like receptor involved in the formation of synapses, is a substrate for presenilin/gamma-secretase-like cleavage. J Biol Chem 277, 49976-49981.
Koudinov, A. R., Berezov, T. T., Kumar, A., and Koudinova, N. V. 1998. Alzheimer's amyloid beta interaction with normal human plasma high density lipoprotein: association with apolipoprotein and lipids. Clin Chim Acta 270, 75-84.
Kuo, Y. M., Emmerling, M. R., Bisgaier, C. L., Essenburg, A. D., Lampert, H. C., Drumm, D., and Roher, A. E. 1998. Elevated low-density lipoprotein in Alzheimer's disease correlates with brain abeta 1-42 levels. Biochem Biophys Res Commun 252, 711-715.
Lee, J. Y., Cole, T. B., Palmiter, R. D., Suh, S. W., and Koh, J. Y. (2002). Contribution by synaptic zinc to the gender-disparate plaque formation in human Swedish mutant APP transgenic mice. Proc Natl Acad Sci USA 99, 7705-7710.
Ling, Y., Morgan, K., and Kalsheker, N. (2003). Amyloid precursor protein (APP) and the biology of proteolytic processing: relevance to Alzheimer's disease. Int J Biochem Cell Biol 35, 1505-1535.
Marambaud, P., Wen, P. H., Dutt, A., Shioi, J., Takashima, A., Siman, R., and Robakis, N. K. (2003). A CBP binding transcriptional repressor produced by the PSI/epsilon-cleavage of N-cadherin is inhibited by PSI FAD mutations. Cell 114, 635-645.
Marzetta, C. A., Savoy, Y. E., Freeman, A. M., Long, C. A., Pettini, J. L., Hagar, R. E., Inskeep, P. B., Davis, K., Stucchi, A. F., Nicolosi, R. J., and et al. (1994). Pharmacological properties of a novel ACAT inhibitor (CP-113,818) in cholesterol-fed rats, hamsters, rabbits, and monkeys. J Lipid Res 35, 1829-1838.
Notkola, I. L., Sulkava, R., Pekkanen, J., Erkinjuntti, T., Ehnholm, C., Kivinen, P., Tuomilehto, J., and Nissinen, A. (1998). Serum total cholesterol, apolipoprotein E epsilon 4 allele, and Alzheimer's disease. Neuroepidemiology 17, 14-20.
Park, I. H., Hwang, E. M., Hong, H. S., Boo, J. H., Oh, S. S., Lee, J., Jung, M. W., Bang, O. Y., Kim, S. U., and Mook-Jung, I. (2003). Lovastatin enhances Abeta production and senile plaque deposition in female Tg2576 mice. Neurobiol Aging 24, 637-643.
Parkin, E. T., Hussain, I., Karran, E. H., Turner, A. J., and Hooper, N. M. 1999. Characterization of detergent-insoluble complexes containing the familial Alzheimer's disease-associated presenilins. J Neurochem 72, 1534-1543.
Petanceska, S. S., DeRosa, S., Olm, V., Diaz, N., Sharma, A., Thomas-Bryant, T., Duff, K., Pappolla, M., and Refolo, L. M. (2002). Statin therapy for Alzheimer's disease: will it work? J Mol Neurosci 19, 155-161.
Puglielli, L., Konopka, G., Pack-Chung, E., Ingano, L. A., Berezovska, O., Hyman, B. T., Chang, T. Y., Tanzi, R. E., and Kovacs, D. M. (2001). Acyl-coenzyme A: cholesterol acyltransferase modulates the generation of the amyloid beta-peptide. Nat Cell Biol 3, 905-912.
Puglielli, L., Tanzi, R. E., and Kovacs, D. M. (2003). Alzheimer's disease: the cholesterol connection. Nat Neurosci 6, 345-351.
Refolo, L. M., Wittenberg, I. S., Friedrich V. L, Jr., and Robakis, N. K. 1991. The Alzheimer amyloid precursor is associated with the detergent-insoluble cytoskeleton. J Neurosci 11, 3888-3897.
Refolo, L. M., Pappolla, M. A., Malester, B., LaFrancois, J., Bryant-Thomas, T., Wang, R., Tint, G. S., Sambamurti, K., and Duff, K. 2000. Hypercholesterolemia accelerates the Alzheimer's amyloid pathology in a transgenic mouse model. Neurobiol Dis 7, 321-331.
Refolo, L. M., Pappolla, M. A., LaFrancois, J., Malester, B., Schmidt, S. D., Thomas-Bryant, T., Tint, G. S., Wang, R., Mercken, M., Petanceska, S. S., and Duff, K. E. (2001). A Cholesterol-Lowering Drug Reduces beta-Amyloid Pathology in a Transgenic Mouse Model of Alzheimer's Disease. Neurobiol Dis 8, 890-899.
Rockenstein, E., Mallory, M., Mante, M., Sisk, A., and Masliah, E. (2001). Early formation of mature amyloid-beta protein deposits in a mutant APP transgenic model depends on levels of Abeta(1-42). J Neurosci Res 66, 573-582.
Saido, T. C. (1998). Alzheimer's disease as proteolytic disorders: anabolism and catabolism of β-amyloid. Neurobiol Aging 19, S69-75.
Schmechel, D. E., Saunders, A. M., Strittmatter, W. J., Crain, B. J., Hulette, C. M., Joo, S. H., Pericak-Vance, M. A., Goldgaber, D., and Roses, A. D. 1993. Increased amyloid 13-peptide deposition in cerebral cortex as a consequence of apolipoprotein E genotype in late-onset Alzheimer disease. Proc Natl Acad Sci USA 90, 9649-9653.
Selkoe, D. J. (1999). Translating cell biology into therapeutic advances in Alzheimer's disease. Nature 399, A23-31.
Shah, P. K. (2003). Emerging non-statin LDL-lowering therapies for dyslipidemia and atherosclerosis. Rev Cardiovasc Med 4, 136-141.
Simons, M., F. Schwarzler, D. Lutjohann, K. von Bergmann, K. Beyreuther, J. Dichgans, H. Wormstall, T. Hartmann, and J. B. Schulz. 2002. Treatment with simvastatin in normocholesterolemic patients with Alzheimer's disease: A 26-week randomized, placebo-controlled, double-blind trial. Ann Neurol. 52:346-50.
Sing, C. F., and Davignon, J. 1985. Role of the apolipoprotein E polymorphism in determining normal plasma lipid and lipoprotein variation. Am J Hum Genet 37, 268-285.
Sinha, S., Anderson, J. P., Barbour, R., Basi, G. S., Caccavello, R., Davis, D., Doan, M., Dovey, H. F., Frigon, N., Hong, J., et al. (1999). Purification and cloning of amyloid precursor protein beta-secretase from human brain. Nature 402, 537-540.
Suzuki, N., Cheung, T. T., Cai, X. D., Odaka, A., Otvos L, Jr., Eckman, C., Golde, T. E., and Younkin, S. G. (1994). An increased percentage of long amyloid β protein secreted by familial amyloid β protein precursor (β APP717) mutants. Science 264, 1336-1340.
Tanzi, R. E., Gusella, J. F., Watkins, P. C., Bruns, G. A., St George-Hyslop, P., Van Keuren, M. L., Patterson, D., Pagan, S., Kurnit, D. M., and Neve, R. L. (1987). Amyloid β protein gene: cDNA, mRNA distribution, and genetic linkage near the Alzheimer locus. Science 235, 880-884.
Vassar, R. (2004). BACE1: the beta-secretase enzyme in Alzheimer's disease. J Mol Neurosci 23, 105-114.
Wang, J., Tanila, H., Puolivali, J., Kadish, I., and van Groen, T. (2003). Gender differences in the amount and deposition of amyloidbeta in APPswe and PS1 double transgenic mice. Neurobiol Dis 14, 318-327.
Wolozin, B., W. Kellman, P. Ruosseau, G. G. Celesia, and G. Siegel. 2000. Decreased prevalence of alzheimer disease associated with 3-hydroxy-3-methyglutaryl coenzyme A reductase inhibitors. Arch Neurol. 57:1439-43.
Wolozin, B. (2004). Cholesterol and the biology of Alzheimer's disease. Neuron 41, 7-10.
Xia, W., and Wolfe, M. S. (2003). Intramembrane proteolysis by presenilin and presenilin-like proteases. J Cell Sci 116, 2839-2844.
   Equivalents

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1. A method for treating an ACAT-related disease in a subject, comprising
administering an ACAT inhibitor using a sustained release delivery system to a subject that has or is suspected of having an ACAT-related disease, wherein the sustained-release implant releases an amount of the ACAT inhibitor for a time that is effective to reduce ACAT activity and thereby treat the ACAT-related disease.

2-4. (canceled)

5. The method of claim 1, wherein the ACAT-related disease is Alzheimer's disease.

6. The method of claim 1, wherein the ACAT-related disease is atherosclerosis.

7. The method of claim 1, wherein the sustained release delivery system is a sustained release implant that is implanted in the subject.

8. The method of claim 1, wherein the sustained release delivery system is a pump-based delivery system.

9. The method of claim 8, wherein the pump-based delivery system is implanted in the subject.

10-13. (canceled)

14. A method for reducing the amount of amyloid-β in a subject, comprising
administering an ACAT inhibitor using a sustained release delivery system to the subject, wherein the sustained release delivery system releases an amount of the ACAT inhibitor for a time that is effective to reduce the amount of amyloid-β in the subject.

15. The method of claim 14, wherein the subject has or is suspected of having elevated levels of amyloid-β, amyloid-β-containing plaque load and/or insoluble amyloid-β.

16. The method of claim 14, wherein the amyloid-β is reduced in the neural tissues of the subject.

17-19. (canceled)

20. The method of claim 14, wherein the subject has or is suspected of having Alzheimer's disease.

21. The method of claim 14, wherein the reduction in amyloid-β in the subject reduces amyloid-β-containing plaque load.

22. The method of claim 14, wherein the reduction in amyloid-β levels is determined by a diagnostic imaging method.

23. The method of claim 14, wherein the sustained release delivery system is a sustained release implant that is implanted in the subject.

24. The method of claim 14, wherein the sustained release delivery system is a pump-based delivery system.

25. The method of claim 24, wherein the pump-based delivery system is implanted in the subject.

26-29. (canceled)

30. A sustained release delivery system configured to contain and deliver to a subject an effective amount of an ACAT inhibitor to inhibit ACAT activity in the subject.

31. The sustained release delivery system of claim 30, wherein the effective amount of the ACAT inhibitor reduces ACAT activity in neural tissues.

32. The sustained release delivery system of claim 30, wherein the effective amount of the ACAT inhibitor reduces amyloid-β levels.

33. The sustained release delivery system of claim 30, wherein the delivery system is an implant.

34. The sustained release delivery system of claim 30, wherein the delivery system is a pump-based system.

35-37. (canceled)
